(19) United States
(12) Patent Application Publication
(10) Pub. No.: US 2005/0187138 A1
(43) Pub. Date: Aug. 25, 2005

(54) Peptide beta-strand mimics based on pyridinones, pyrazinones, pyridazinones, and triazinones

(75) Inventors: Paul A. Bartlett, Oakland, CA (US); Ming Chen Hammond, Alameda, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: THE REGENTS OF THE UNIVERSITY OF CALIFORNIA, a California corporation, Oakland, CA (US)

(21) Appl. No.: 10/786,615

(22) Filed: Feb. 24, 2004

(51) Int. Cl. A61K 38/00

(52) U.S. Cl. 514/2; 530/409

(57) ABSTRACT

Peptide analogs in which one or more amino acids is replaced by a diaza- or triazacyclohexenone, or by an aza-, diaza-, or triazacyclohexenone that is substituted at the α-position with a side chain of an amino acid, display an improved ability to assume a β-strand conformation and to enter into β-sheet-like interactions with peptides in an affinity-specific manner. The peptide analogs of this invention therefore have utility as β-strand mimics offering advantages over both native peptides and β-strand mimics of the prior art.

### STATEMENT AS TO RIGHTS TO INVENTIONS MADE UNDER FEDERALLY SPONSORED RESEARCH AND DEVELOPMENT

This invention was made with government support under Grant No. GM-30759 awarded by the National Institutes of Health. The Government has certain rights to this invention.

### BACKGROUND OF THE INVENTION

1. Field of the Invention

This invention resides in the field of proteins and the complexations and interactions of proteins with other proteins and with nucleic acids through β-sheet interactions. The particular areas addressed by this invention are compositions for and methods of modifying the ability of proteins to enter into these interactions and the various benefits that are derived from such modifications, including changes to the biological activity of the proteins.

2. Description of the Prior Art

The conformation of proteins and peptides is largely governed by secondary structural elements, such as α-helices, β-turns, and β-strands, which determine the three-dimensional orientation of the amino acid side chains and thereby the longer range interstrand and intermolecular interactions. Both β-strands and the β-sheets derived from β-strands play important roles in protein-protein interactions as well as the association of proteins with other biopolymers such as ribosomal RNA and nucleic acids. Disclosures of these roles are found in Fitzgerald, F. M. D., et al., J. Biol. Chem. 1990, vol. 265, 14209; Zutshi, R., et al., J. Am. Chem. Soc. 1997, vol. 119, 4841; Babe, L. M., et al., Protein Sci. 1992, vol. 1, 1244; Siligardi, G., et al., Biopolymers (peptide science) 1995, vol. 37, 281; Buckle, A. M., et al., Proc. Natl. Acad. Sci. USA 1997, vol. 94, 3571; Taneja, B. C., et al., Protein Engineering 1999, vol. 12, 815; Stern, L. J., et al., Nature 1994, vol. 368, 215; Moss, N., et al., J. Med. Chem. 1996, vol. 39, 2178; Sauer, F. G., et al., Science 1999, vol. 285, 1058; Karlsson, K. F., et al., J Bioorg. Med. Chem. 1998, vol. 6, 2085; Maitra, S.; Nowick, J. S. In The Amide Linkage; Greenberg, A.; Breneman, C. M.; Liebman, J. F.; Eds.; John Wiley & Sons, Inc.: New York, N.Y., 2000; pp 495-518; Lynn, D. G., et al., J. Struc. Biol. 2000, vol. 130, 153; Wilkinson, A. J. Chem. Biol. 1996, vol. 3, 519; Jones, S., Thornton, J. M. Proc. Natl. Acad. Sci. USA 1996, vol. 93, 13; Tateno, M., et al., Biopolymers 1998, vol. 44, 335; Connolly, K. M., et al., J. Mol. Biol. 2000, vol. 300, 84; Cohen, S. X., et al., EMBO J. 2003, vol. 22, 1835. For example, the β-sheet-like association and precipitation of hydrophobic protein fragments in amyloid plaques is strongly implicated in neurodegenerative diseases, as disclosed by Cohen, F. E.; Kelly, J. W. Nature 2003, vol. 426, 905; Roloff, E. V., et al., Cell. Mol. Life Sci. 1999, vol. 55, 601; Yatin, S. M., et al., J. Mol. Neurosci. 1998, vol. 11, 183; and Prusiner, S. B., et al., Cell 1998, vol. 93, 337. Furthermore, various biological processes depend on the accessibility of individual peptide strands. Examples of these processes are:
vancomycin complexation of the Lys-D-Ala-D-Ala peptide in bacterial cell wall synthesis;
homodimerization of HIV protease, which involves a “fireman's grip” β-sheet interaction among the N-terminal residues;
heterodimerization of ribonucleotide reductase and HIV reverse transcriptase, which can be blocked with soluble oligopeptides corresponding to part of the interface regions;
dimerization of the λ-Cro repressor via an antiparallel β-strand; and
protein-protein association via PDZ domains.

Systems that mimic and block these interactions are disclosed by Smith, A. B., et al., J. Am. Chem. Soc. 1992, vol. 114, 10672; Smith, A. B., et al., J. Am. Chem. Soc. 1994, vol. 116, 9947-9962; Smith, A. B., et al., J. Am. Chem. Soc. 1995, vol. 117, 11113-11123; Smith, A. B., et al., Bioorg. Med. Chem. 1996, vol. 4, 1021; Smith, A. B., et al., J. Am. Chem. Soc. 1999, vol. 121, 9286-9298; Smith, A. B., et al., Organic Letters 2000, vol. 2, 2037; Smith, A. B., et al., Organic Letters 2000, vol. 2, 2041; Hirschmann, R., et al, U.S. Pat. No. 5,489,692, issued Feb. 6, 1996; Hirschmann, R. F., et al, U.S. Pat. No. 5,514,814, issued May 7, 1996; Hirschmann, R. F., et al, U.S. Pat. No. 5,770,732, issued Jun. 23, 1998; Smith, III, A. B., et al, U.S. Pat. No. 6,034,247, issued Mar. 7, 2000; Nowick, J. S., et al., J. Am. Chem. Soc. 2000, vol. 122, 654-661; Nowick, J. S., et al., J. Am. Chem. Soc. 2001, vol. 123, 5176-5180; Nowick, J., et al., International Patent Application No. WO 01/14412, published Mar. 1, 2001, under the Patent Cooperation Treaty; Nowick, J. S., Chung, D. M. Angew. Chem. Int. Ed. 2003, vol. 42, 1765; Boumendjel, A., Roberts, J. C., Hu, E., Pallai, P. V., Rebek, J., J. Org. Chem. 1996, vol. 61, 4434; and Kemp, D. S., et al., J. Org. Chem. 1990, vol. 55, 4650-7.

The effectiveness and utility of these mimicking systems is enhanced by incorporating sequence selectivity into the structures. Sequence-selective peptide complexing agents that interact with extended peptides or with exposed loops on proteins offer potential as biological tools and therapeutic approaches, as well as serving as the basis for a proteomics equivalent of gene expression analysis.

Reported investigations of sequence-selective peptide complexing agents range from non-peptidic cavitands disclosed by Still, W. C., Acc. Chem. Res. 1996, vol. 29, 155-163, Chen, C.-T., Wagner, H., Still, W. C., Science 1997, vol. 279, 851; Malteic, M., et al., Angew. Chem. Int. Ed. 1996, vol. 35, 1490, to highly flexible structures in which two peptide or peptide-like strands are connected by a variety of linkers, reported for example by LaBrenz, S. R., et al., J. Am. Chem. Soc. 1995, vol. 117, 1655-1656; Lowik, D. W. P. M., et al., Angew. Chem., Int. Ed. 1998, vol. 37, 1846-1850; Hioki, H., et al., Tetrahedron Lett. 1999, vol. 40, 6821-6825; Botana, E., et al., Chem. Commun. 2001, 1358; Braxmeier, T., et al., Chem. Eur. J. 2001, vol. 7, 1889; Davies, M., et al., J. Org. Chem. 1998, vol. 63, 8696; Fessmann, T., et al. Angew. Chem., Internat. Edit. Engl. 1999, vol. 38, 1993; Wennemers, H., et al., Chem. Eur. J. 2001, vol. 7, 3342; Bouras, A., et al., J. Med. Chem. 1999, vol. 42, 957; Song, M., et al., Bioorg. Med. Chem. Lett. 2001, vol. 11, 2465; Boyce, R., et al., J. Am. Chem. Soc. 1994, vol. 116, 7955; Cheng, Y., Senaga, T., Still, W. C., J. Am. Chem. Soc. 1996, vol. 118, 1813; Ghosh, I., Chmielewski, J Chemistry & Biology 1998, vol. 5, 439; and Zutshi, R.; Chmielewski, J. Bioorg. Med. Chem. Lett. 2000, vol. 10, 1901. The host-guest complex produced by these highly flexible structures is a three-stranded β-sheet whose outer strands provide both affinity through the hydrogen-bonding network and selectivity through interactions between side chains. The success of this approach has been limited however for several reasons. One reason is that the design fails to include bridging units that provide precise positioning of the side arms. Another is that the high flexibility of the peptide arms causes formation of the desired complex to be disfavored entropically. A third is that hydrogen bonding, while favorable in organic solvents, is not a strong binding force in water in the absence of pre-organization. For those host-guest pairs that have sufficient hydrophobic binding to assemble in water, the high degree of hydrophobic binding tends to cause them to aggregate further.

All published materials cited throughout this specification are hereby incorporated herein by reference.

### SUMMARY OF THE INVENTION

It has now been discovered that 1,6-dihydro-3(2H)-pyridinones that are substituted in the 6-position (designated the “α-position”) with an amino acid side chain are highly effective when used as units in peptide β-strand mimics, and that related heterocycles whose 2- and 4-position ring vertices are replaced by additional N atoms (or NH groups), including those that are substituted with an amino acid side chain at the α-position, are also highly effective as units in peptide β-strand mimics. Thus, four classes of structures are disclosed herein as amino acid substitutes in peptide analogs. These four classes are:
(1) α-substituted (i.e., 6-substituted) 1,6-dihydro-3(2H)-pyridinones,
(2) 4,5-dihydro-2(3H)-pyrazinones and α-substituted (i.e., 5-substituted) 4,5-dihydro-2(3H)-pyrazinones,
(3) 1,6-dihydro-3(2H)-pyridazinones and α-substituted (i.e., 6-substituted) 1,6-dihydro-3(2H)-pyridazinones, and
(4) 1,6-dihydro-1,2,4-triazin 3(2H)-ones and α-substituted (i.e., 6-substituted) 1,6-dihydro-1,2,4-triazin 3(2H)-ones,
   in which the α-substituent, in those species where such a substituent is indicated, is an amino acid side chain. Collectively, these four classes are referred to herein as “azacyclohexenones,” the prefix “aza” denoting either one, two, or three nitrogen atoms. The term “azacyclohexenones” is used herein to encompass both those species that are unsubstituted at the α-position and those that bear an amino acid side chain at the α-position, the latter subset being referred to occasionally herein as “α-substituted azacyclohexenones.” Peptide analogs in which at least one but less than all amino acids is replaced by an azacyclohexenone unit or an α-substituted azacyclohexenone unit of the present invention readily enter into β-sheet-like interactions of particularly high strength, and those that are α-substituted enter into β-sheet-like interactions that are even more sequence-specific due to the amino acid side chain(s) at the α-position of the unit(s). The present invention also extends to peptide analogs in which one or more amino acids are replaced by any one of the azacyclohexenones described above and one or more additional amino acids are replaced by 1,6-dihydro-3(2H)-pyridinones that bear no α-substituent, such as those disclosed in United States Published Patent Application No. U.S. 2003-0073721 A1, published Apr. 17, 2003.

Also encompassed by this invention are hybrid constructs that consist of a peptide analog sequence covalently linked to another peptide analog sequence or to a conventional peptide sequence. In the peptide analog sequences, at least one but not all amino acids is replaced by an azacyclohexenone unit of the present invention. The linkage between the two sequences may consist of amino acid units that connect the sequences in a head-to-tail orientation and that permit a β-turn, in which case the peptide analog sequence, by virtue of its strong tendency to enter into stable β-sheet-like interactions with conventional peptide and other peptide analog sequences, forms a β-hairpin that stabilizes the other sequence in an extended β-strand conformation. The linkage between the two sequences may consist of other connecting units than amino acids. Moreover, the linkage may connect the sequences in a head-to-head or a tail-to-tail orientation so that the resulting molecule can form a complex with a separate conventional amino acid sequence. Examples of such linkages are found in the prior art related to sequence-selective peptide complexation (cited above). The present invention is not however limited to those specific linkages.

The peptide analogs and peptide-analog hybrids of this invention have many applications. They can serve for example as tools for studying β-sheet nucleation, propagation, and suppression. They can also serve as prophylactic or palliative agents in physiological conditions that involve or are controllable by β-sheet interactions. For example, these peptide analogs and hybrids can be used in the treatment of prion diseases such as “mad cow disease” and other neurodegenerative diseases such as Alzheimer's disease which arise from the association of certain hydrophobic proteins to form insoluble (3-sheet aggregates known as amyloid complexes. This utility arises from the enhanced ability of the analogs and hybrids of this invention to bind to an exposed surface of the amyloid β-sheet complex and prevent further aggregation. The peptide analogs and hybrids can also be used for blocking the infectivity of the human immunodeficiency virus by inhibiting the association of the viral gp 120 protein with the CD4 receptor on the T-lymphocyte cell surface. A still further use is the blocking of the effects of inflammatory chemokines that are involved in allergic reactions, psoriasis, arthritis, atherosclerosis, multiple sclerosis, and cancer.

Peptide analogs in accordance with this invention function similarly to antibodies by binding to peptides and proteins in a sequence-selective manner. As such, the peptide analogs and peptide-analog hybrids of this invention are useful for example as protein purification media in affinity chromatography. They are also useful as components in diagnostic devices or kits, where they can be used for the concentration and identification of peptide and protein analytes. This antibody-type character also provides utility in vivo, where the peptide analogs and peptide-analog hybrids can be used for therapeutic effects by forming complexes with and blocking the action of specific peptide hormones or by targeting attached radiopharmaceuticals or cytotoxic agents to specific sites in the body. A collection of peptide analogs and hybrids in accordance with this invention can be arranged in an array such as that of a proteomics chip for use in an assay for the levels of expression of specific proteins in different tissues and under different conditions. Other uses will be readily apparent to those skilled in the art.

The present invention thus resides in:
Azacyclohexenones in which the prefix “aza” denotes that one, two, or three carbon atoms in the cyclohexenone ring are replaced by a nitrogen atom, including α-substituted azacyclohexenones in which “α-substituted” denotes that the ring carbon opposite to the carbonyl group is substituted with an amino acid side chain, the azacyclohexenones, α-substituted or otherwise, being either functionalized for linkage to each other or to amino acids by formation of carbon-nitrogen bonds, or covalently bonded to one or more amino acids through carbon-nitrogen bonds
Peptide analogs in which at least one amino acid, but not all, is replaced by an azacyclohexenone group, α-substituted or otherwise, as described above
Peptide-analog hybrids consisting of peptide analogs covalently linked to other peptide analogs or to conventional peptides as described above, in head-to-tail, head-to-head, or tail-to-tail orientation
The use of peptide analogs and peptide-analog hybrids as described above for inhibiting β-sheet-like interactions between proteins
The use of peptide analogs and peptide-analog hybrids as described above for inhibiting the biological activity of a peptide or protein
The use of peptide analogs and peptide-analog hybrids as described above for extracting a target peptide from a mixture of peptides or a target protein from a mixture of proteins

Other structures, uses, embodiments, applications and features of the invention will be apparent from the description that follows.

### BRIEF DESCRIPTION OF THE DRAWINGS

FIG. 1 is an illustrative reaction scheme for one subclass of constructs within the scope of this invention.

FIG. 2 is an illustrative reaction scheme for a second subclass of constructs within the scope of this invention.

FIG. 3 is an illustrative reaction scheme for a third subclass of constructs within the scope of this invention.

### DETAILED DESCRIPTION OF THE INVENTION AND PREFERRED EMBODIMENTS

Definitions

The term “β-strand conformation” is used herein to denote the three-dimensional conformation of a single peptide strand in which the strand is elongated such that its amide groups form a planar zig-zag backbone and the amino acid side chains extend out of the plane to either or both sides. A peptide strand may assume this conformation either on its own or in combination with another peptide (or peptide analog) in a β-sheet-like interaction as defined below.

The term “β-sheet-like interaction” is used herein to denote the interaction between two peptides both of which are in a β-strand conformation, in which the two strands are held together side-by-side with hydrogen bonding between carbonyl oxygens in one backbone and amide NH groups in the other (and vice versa). The orientation of the strands may be parallel or anti-parallel, although the anti-parallel relationship is preferred. The term also extends to the analogous interaction that occurs when one of the peptides is replaced by a peptide analog or another elongated molecule in which similar hydrogen bonds are formed along the lengths of the molecules. Any peptide analog in accordance with this invention may thus enter into a β-sheet-like interaction with a peptide, with itself, or with another peptide analog. An individual peptide may engage in β-sheet-like interactions with two such peptides, analogs or other molecules, one on each side of the first peptide.

The term “β-turn” is used herein to denote a sharp 180-degree (“hair-pin”) turn in a peptide sequence that places the segments on either side of the turn in sufficient proximity to engage in hydrogen bonding between opposing units in the segments such that the segments align to form a β-sheet-like interaction. In recitations of a linkage that “permits . . . a β-sheet-like interaction,” “permits a hair-pin turn,” and similar phrases, the word “permit” denotes that the linkage is capable of adopting a β-turn conformation with little or no resistance, as opposed to linkages that offer steric or electronic resistance to adopting a β-turn conformation.

The term “peptide” is used herein to denote a compound containing two or more of amino acid residues joined by an amide bond formed from the carboxyl group of one residue and the amino group of the adjacent residue. The term “amino acid” includes both naturally occurring and synthetic amino acids, as well as amino acid analogs and amino acid mimetics whose properties are similar to those of the naturally occurring amino acids. Naturally occurring amino acids are those that are encoded by the genetic code, as well as those that are modified after expression, such as hydroxyproline, carboxyglutamate, O-phosphoserine, and glycosylated amino acids. Amino acid analogs are compounds having functionalities similar to those of naturally occurring amino acids, i.e., an amino group, a carboxyl group, and one or more side chains attached to a framework of from 1 to 4 carbon atoms. Many such analogs are known to those skilled in the art, including but not limited to homoserine, norleucine, methionine sulfoximine, phenylglycine, (p-fluorophenyl)alanine, β-alanine, α-aminoisobutyric acid, tert-leucine, and β-methylaspartic acid. The term “amino acid mimetic” is used herein to denote a moiety other than a naturally occurring amino acid that conformationally and functionally serves as a substitute for an amino acid in a peptide while still allowing the peptide to enter into β-sheet interactions. A variety of amino acid mimetics is listed by Morgan, B. A., and J. A. Gainor, Ann. Rep. Med. Chem., vol. 24, 243-252 (1989). The term “peptide” includes, but is not limited to, proteins.

The term “amino acid side chain” denotes the group represented by the “R” in the amino acid formula 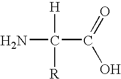
and includes any of the side chains in naturally occurring amino acids as well as those in modified amino acids and amino acid mimetics, and includes side chains in protected as well as unprotected form. While glycine is an amino acid having an H atom in the R position, the term “amino acid side chain” as used herein does not include H. In general, preferred amino acid side chains are C₁-C₆ alkyl, C₁-C₆ alkyl interrupted by —O—, C₁-C₆ alkyl interrupted by —S—, hydroxy-(C₁-C₆ alkyl), carboxy-(C₁-C₆ alkyl), amino-(C₁-C₆ alkyl), guanidino-(C₁-C₆ alkyl), carbamoyl-(C₁-C₆ alkyl), mercapto-(C₁-C₆ alkyl), indolyl-(C₁-C₃ alkyl), phenyl-(C₁-C₃ alkyl), hydroxyphenyl-(C₁-C₆ alkyl), halophenyl-(C₁-C₆ alkyl), imidazolyl-(C₁-C₆ alkyl), phenyl, and sulfoximino-(C₁-C₆ alkyl). The most preferred are C₁-C₄ alkyl, hydroxy-(C₁-C₂ alkyl), carboxy-(C₁-C₂ alkyl), amino-(C₃-C₅ alkyl), guanidino-(C₂-C₄ alkyl), carbamoyl-(C₁-C₂ alkyl), mercapto-(C₁-C₂ alkyl), methylthio-(C₁-C₃ alkyl), indolylmethyl, phenyl-(C₁-C₂ alkyl), and hydroxyphenyl-(C₁-C₂ alkyl).

The term “activated leaving group” is used herein to denote a radical or group of atoms that is displaced from a carbon atom by the attack of a nucleophile in a nucleophilic substitution reaction.

The term “protecting group” is used herein to denote a radical or group of atoms that is bound to a particular atom of a molecule to prevent that atom from participating in reactions occurring on other portions of the molecule.

The term “amine protecting group” is used herein to denote a radical or group of atoms that is bound to an amine nitrogen atom of a molecule to prevent that nitrogen atom from participating in reactions occurring on other portions of the molecule. The term “amine-protected” denotes the structural characteristic of a molecule containing an amine nitrogen atom by which that nitrogen atom is prevented from participating in reactions occurring on other portions of the molecule.

The term “carboxy protecting group” is used herein to denote a radical or group of atoms that is bound to a carboxy oxygen atom of a molecule to prevent that oxygen atom from participating in subsequent reactions occurring on other portions of the molecule. The term “carboxy-protected” denotes the structural characteristic of a molecule containing a carboxy oxygen atom by which that oxygen atom is prevented from participating in reactions occurring on other portions of the molecule.

The term “side chain protecting group” is used herein to denote a radical or group of atoms that is bound to a nitrogen atom or an oxygen atom or a sulfur atom contained within an amino acid side chain or within the α-substituent of an azacyclohexenone to prevent that side chain functionality from participating in reactions occurring on other portions of that molecule. The term “side chain-protected” denotes the structural characteristic of a molecule containing a side chain functionality by which that side chain functionality is prevented from participating in reactions occurring on other portions of the molecule.

The term “solid support” is used herein to denote any inert solid that can be used to facilitate the separation of bound species from free species in the course of a solid-supported synthesis or in a binding interaction such as a chromatographic separation or any of various analytical procedures that involve affinity-type binding. Solid supports include particles such as those used in chromatography columns as well as the inner walls of reaction vessels such as test tubes and the wells of microtiter plates, and other configurations well known to clinicians and laboratory technicians. Examples of materials used as solid supports are agarose, polystyrene, polyacrylamide, and these materials modified by poly(ethylene glycol). A peptide analog can be attached to these supports through the C-terminus (for example by an ester or amide linkage), through the N-terminus (for example, by a urea or carbamate linkage), or through a functionalized side chain (for example, by ester, amide, urea, carbamate, disulfide, or ether linkages).

Compounds, Peptide Analogs and Constructs of the Invention

The generic formula for the azacyclohexenone unit included in the compounds, analogs, and constructs of this invention, is the structure shown between in the brackets of the following formula:

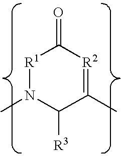

In this formula, R¹ is CH₂ or NH, R² is CH or N, and for those structures in which R¹ is CH₂ and R² is CH, R³ is an amino acid side chain, while for those structures in which R¹ is NH, or R² is N, or both R¹ is NH and R² is N, R³ is either H or an amino acid side chain. When two or more of these units are present in a peptide analog, hybrid, or other molecular structure in accordance with this invention, R¹, R², and R³ of any one of these units in the structure are either the same as or different from R¹, R², and R³ of any other of these units in the structure. Of the four structurally distinct classes of these units recited above, 1,6-dihydro-3(2H)-pyridinones are those in which R¹ is CH₂ and R² is CH, 4,5-dihydro-2(3H)-pyrazinones are those in which R¹ is CH₂ and R² is N, 1,6-dihydro-3(2H)-pyridazinones are those in which R¹ is NH and R² is CH, 1,6-dihydro-1,2,4-triazin 3(2H)-ones are those in which R¹ is NH and R² is N, and those that are α-substituted bear an amino acid side chain (as defined above to mean substituents other than H itself) at the 6-position in 1,6-dihydro-3(2H)-pyridinones, 1,6-dihydro-3 (2H)-pyridazinones, and 1,6-dihydro-1,2,4-triazin 3(2H)-ones, and at the 5-position in 4,5-dihydro-2(3H)-pyrazinones. The pyrazinones, i.e. are those in which R¹ is CH₂ and R² is N, substituted or unsubstituted at the α-position, are preferred.

The α-substituted azacyclohexenone derivatives (i.e., those in which R³ is an amino acid side chain) exist in two stereoisomeric forms. The enantiomers of the α-substituted azacyclohexenones that correspond to the L configuration of the natural amino acids are the more desirable isomers because peptide analogs incorporating these enantiomers enter into β-sheet-like interactions with natural peptides and proteins more readily and with higher affinity and selectivity than peptide analogs incorporating α-substituted azacyclohexenones of the opposite configuration.

The functionalized azacyclohexenones of this invention are those having the formula 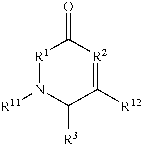
in which R¹¹ is a nitrogen protecting group, and R¹² is either OH, SH, or an activated leaving group. As those skilled in the art will recognize, these structures are capable of other tautomeric forms. Such tautomeric forms are likewise within the scope of this invention.

Peptide analogs of this invention include compounds of the following formulas: 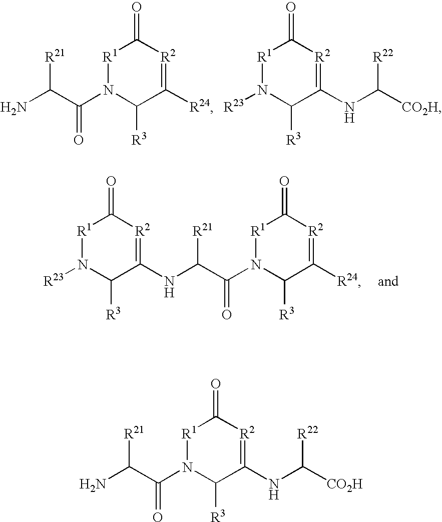
in which:
R²¹ is H or an amino acid side chain,
R²² is H or an amino acid side chain and may be the same as or different from R²¹ when both appear in the same compound,
R²³ is either H or an amine protecting group, and
R²⁴ is either:
   an activated leaving group,
   OR²⁵ where R²⁵ is H or an oxygen-protecting group,
   SR²⁶ where R²⁶ is H or an alkyl or aryl group, or
   N(R²⁷)₂ where the R²⁷'s are independently H, alkyl, or aryl.

Also included are amine-protected analogs of the compounds that terminate in H₂N—, carboxy-protected analogs of the compounds that terminate in —CO₂H, carboxy-activated analogs of the compounds that terminate in —CO₂H, amine-protected and carboxy-protected analogs of 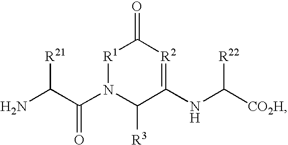
and amine-protected and carboxy-activated analogs of 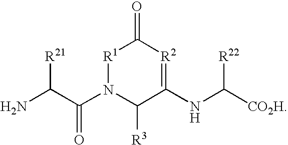
When two azacyclohexenone units are present, the R¹, R², and R³ groups may vary independently.

Among the peptide analogs listed above, one preferred subclass is that defined by the formula 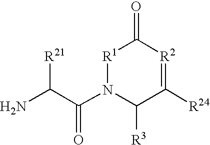
in which R²⁴ is an activated leaving group, or OR²⁵ where R²⁵ is H or an oxygen-protecting group, or SR²⁶ where R²⁶ is H or an alkyl or aryl group. This subclass also includes amine-protected analogs of this formula.

Another preferred subclass is that defined by the formula 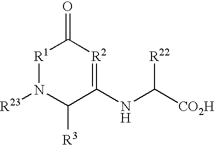
in which R²³ is an amine protecting group, including carboxy-protected analogs of this formula.

A further preferred subclass is that defined by the formula 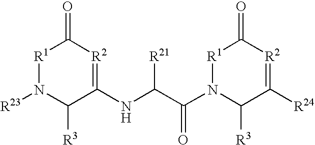
in which R²³ is an amine protecting group and R²⁴ is either an activated leaving group, OR²⁵ where R²⁵ is H or an oxygen-protecting group, or SR²⁶ where R²⁶ is H or an alkyl or aryl group.

A still further preferred subclass is that defined by the formula 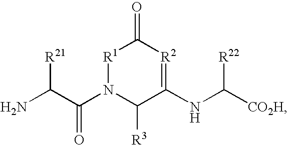
including amine-protected analogs, carboxy-protected analogs, amine-protected and carboxy-protected analogs, and amine-protected and carboxy-activated analogs of this formula.

The peptide analogs within these preferred classes are useful as components in the synthesis of longer-chain peptide analogs. In certain embodiments of this invention, the R²¹ and R²² groups in these formulas are side chains, or protected side chains, of natural amino acids. In other embodiments, either R²¹, R²², or both are side chains, or protected side chains, of unnatural amino acids.

Further peptide analogs of this invention are defined as peptides in which at least one amino acid, but less than all amino acids, is replaced by an azacyclohexenone group described above. Preferred analogs are those containing from 2 to 200 amino acids and from 1 to 100 azacyclohexenone groups. More preferred are those that contain from 2 to 100 amino acids and from 1 to 50 azacyclohexenone groups, and most preferred are those that contain from 2 to 10 amino acids and from 1 to 20 azacyclohexenone groups. The number ratio of azacyclohexenone groups to amino acids in these analogs is preferably from 1:10 to 10:1, more preferably from 1:5 to 5:1, and most preferably from 1:2 to 1:1.

The remaining amino acids in the peptide analogs of this invention, i.e., those that have not been replaced by the azacyclohexenone units described above, can include any natural amino acids, amino acid analogs, amino acid mimetics, or any combination of these three groups. Preferably, the remaining amino acids are either all natural amino acids or a combination of natural and unnatural amino acids. Particularly preferred amino acids are those whose side chains are members of the preferred groups listed in the definition of “amino acid side chain” above.

In peptide analogs of this invention in which two or more amino acids are replaced by azacyclohexenone units, it is preferred that the azacyclohexenone units occupy non-adjacent positions along the peptide chain. In certain embodiments, every second amino acid is replaced by a corresponding α-substituted azacyclohexenone unit. An α-substituted azacyclohexenone unit is termed “corresponding” when the R³ group is the same amino acid side chain as that of the amino acid that the α-substituted azacyclohexenone unit replaces.

Still further peptide analogs of this invention are those defined by the following formula 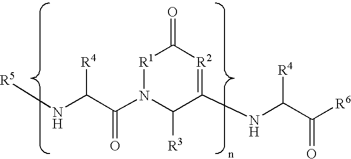
in which:
the R⁴'s are the same or different and each R⁴ is H or an amino acid side chain;
R⁵ is either a peptide chain terminating group or 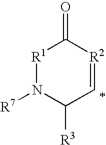
   in which R⁷ is either H, alkyl, acyl, carbamoyl, or alkoxycarbonyl, and * denotes the site of attachment;
   R⁶ is either a peptide chain terminating group or 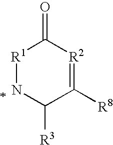
   in which R⁸ is either hydroxyl, alkoxy, mercapto, alkylmercapto, amino, alkylamino, dialkylamino, or arylamino, and * denotes the site of attachment; and
   n is at least 2.

Preferred subclasses among these peptide analogs are those in which the R⁴'s are a combination of side chains of natural and unnatural amino acids, and optionally H as well, and those in which each R⁴ is either H or a side chain of a natural amino acid. Further preferred subclasses are those in which R⁵ is either H, alkyl, acyl, carbamoyl, or alkoxycarbonyl. A still further preferred subclass is that in which R⁵ is

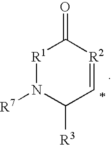

In terms of the R⁶ group, a preferred subclass is that in which R⁶ is either hydroxyl, alkoxyl, amino, alkylamino, dialkylamino, or arylamino, with hydroxyl and alkylamino (particularly C₁-C₄ alkylamino) most preferred. A still further preferred subclass is that in which R⁶ is

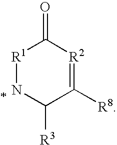

Among the peptide analogs containing the symbol “n” as an index of chain length, a preferred subclass is that in which n is 2 to 100, a more preferred subclass is that in which n is 2 to 50, and a most preferred subclass is that in which n is 2 to 5.

Constructs or hybrids in accordance with this invention include two sequences linked together by a linkage that permits a βturn, the first sequence being a sequence of amino acids joined together by amide bonds as in a conventional peptide, and the second sequence being a sequence of amino acids joined together by amide bonds as in the first sequence except that one or more, but not all, of the amino acids is replaced by an azacyclohexenone unit of the present invention. The azacyclohexenone unit(s), with the assistance of the covalent linkage, induces a β-sheet interaction between the two sequences and thereby induces and stabilizes the first sequence, i.e., the all-amino-acid sequence, in a β-strand conformation. In this configuration, the all-amino acid sequence is particularly effective in engaging in β-sheet interactions with other (“target”) peptides and thus performing such functions as inhibiting the target peptides from entering into β-sheet interactions with further peptides and thereby inhibiting the biological activity of these target peptides, and various affinity-type functions such as extracting the target peptides from peptide mixtures or mixtures in general. The linkage can also assist the sequences to enter into a (3-sheet interaction with a third peptide sequence and thereby inhibit the biological activity of the third sequence. In either case, the construct size (i.e., the lengths of the two segments) is not critical to the invention, but in preferred embodiments, the all-amino-acid segment will contain from 3 to 200 amino acids and in the segment containing both amino acids and azacyclohexenone units the total of the amino acids and azacyclohexenone units will range from 3 to 200. Ranges for both segments that are more preferred are 3 to 100, and most preferred are 3 to 20. The linkage between the segments can vary and is not critical to the invention except that, for segments that are linked head-to-tail by amino acid units, the linkage should not be one that is sterically or otherwise hindered from assuming a β-turn conformation. Preferred linkages are those that favorably assume or promote a β-turn conformation. Examples are D-proline-alanine (D-Pro-Ala) and asparagine-glycine (Asn-Gly).

In the constructs of this invention as well as the peptide analogs that contain α-substituted azacyclohexenone units and are intended to enter into β-sheet-like interactions with target peptides, the amino acids of the α-substituted azacyclohexenone-containing sequence are preferably those whose side chains are chosen on the basis of known side chain-side chain affinities within β-sheets through design of sterically and electronically complementary structures, or by screening analogs. Likewise, the R³ groups of the α-substituted azacyclohexenone units are preferably chosen on the basis of their affinities with the side chains of opposing amino acids in the β-sheet conformation. These affinities and their role in β-sheet-like interactions are known in the art. See, for example, Smith, C. K., et al., “Guidelines for Protein Design: The Energetics of (3-Sheet Side Chain Interactions,” Science 1995, vol. 270, 980; Ramirez-Alvarado, M., et al., “De novo design and structural analysis of a model β-hairpin peptide system,” Nature Structural Biology 1996, vol. 3, 604; von Heijne, G., et al., “The β-Structure: Inter-Strand Correlations,” J. Mol. Biol. 1997, vol. 117, 821. A further method for selecting the amino acids and the R³ groups of the azacyclohexenone-containing sequence is to base the selection on a peptide sequence known to be complementary to the target peptide. Thus, in accordance with known principles, the side chains of the amino acids in the α-substituted azacyclohexenone-containing sequence preferably do not repel, but are instead compatible with, the side chains of the amino acids at the corresponding locations of the all-amino-acid segments or target peptides or proteins. This complementarity may result from a pairing of directly opposing residues but the affinity of any particular residue for an opposing residue may also be influenced by neighboring residues. Some of the ways in which directly opposing residues can be selected to achieve compatibility are the inclusion of basic side chains in the α-substituted azacyclohexenone-containing sequence to oppose acidic side chains in the conventional peptide (all-amino-acid) sequence, acidic side chains in the α-substituted azacyclohexenone-containing sequence to oppose basic side chains in the conventional peptide sequence, hydrophobic side chains in one sequence to oppose hydrophobic side chains in the other sequence, and hydrophilic side chains in the one sequence to oppose hydrophilic side chains in the other sequence. The characters of the side chains of known amino acids are well known to those skilled in the art and hence the appropriate selection for optimal favoring of β-sheet interaction will be readily apparent on this basis. The following is a rough characterization of several amino acids:

| Side Chain Character | Amino Acids |
|---|---|
| acidic | aspartic acid, glutamic acid |
| basic | arginine, histidine, lysine |
| hydrophobic | alanine, isoleucine, leucine, methionine, |
| | phenylalanine, valine, tryptophan, tyrosine |
| hydrophilic | asparagine, glutamine, serine, threonine |

Synthesis of the Compounds, Peptide Analogs, and Constructs of the Invention

Abbreviations used in the following synthesis descriptions and examples are as follows:

| | |
|---|---|
| Ac | acetyl |
| Alloc | allyloxycarbonyl |
| Boc | t-butoxycarbonyl |
| Cbz | benzyloxycarbonyl |
| DCM | dichloromethane |
| DIEA | diisopropylethylamine |
| DMF | dimethylformamide |
| DMSO | dimethylsulfoxide |
| EtOAc | ethyl acetate |
| EtOH | ethanol |
| HATU | N-[(dimethyl-amino)-1H-1,2,3-triazolo- |
| | [4,5-b]pyridin-1-yl-methylene]-N- |
| | methylmethanaminium hexafluoro- |
| | phosphate N-oxide |
| iPr | isopropyl |
| LDA | lithium diisopropylamide |
| MeCN | acetonitrile |
| MeOH | methanol |
| Pf | 9-phenyl-9-fluorenyl |
| Ph | phenyl |
| tBu | t-butyl |
| TEA | triethylamine |
| TFA | trifluoroacetic acid |
| THF | tetrahydrofuran |
| TLC | thin-layer chromatography |

The azacyclohexenones and their functionalized derivatives can be synthesized by conventional methods. The dihydropyrazinone (in which R¹ is CH₂ and R² is N), for example, can be prepared from an amide of an amino acid whose side chain is the same as, or a protected version of, R³ sought for the product. The amide is thionated preferentially relative to the carbamate and ester groups with Lawesson's reagent using known methods as reported by Cava, M. P., et al., Tetrahedron 1985, vol. 41, 5061-5087. The thioamide can then be cyclized by base-induced cyclization while protecting the stereocenter, as reported by Koehler, W., et al., Chem. Ber. 1967, vol. 100, 1073-1081. The cyclic thioamide is then activated by S-methylation, followed by reaction with an amino acid or peptide derivative to give the dihydropyrazinone peptide analog. This reaction scheme is shown below:

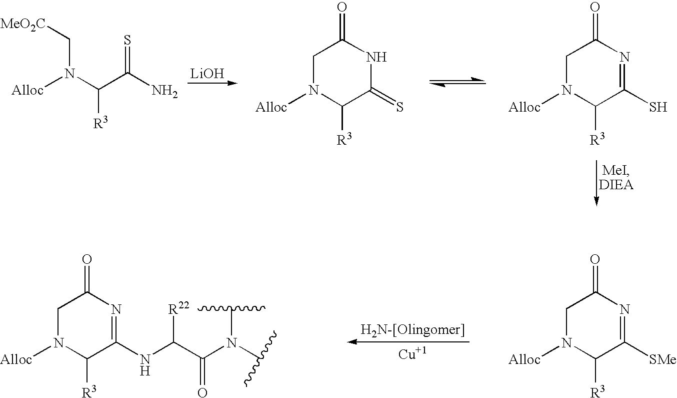

In an alternative route, the amide is first converted to a thioimidate salt which is then converted to the amidine under neutral conditions. The amidine can then be cyclized at the less hindered nitrogen to give the dihydropyrazinone peptide analog.

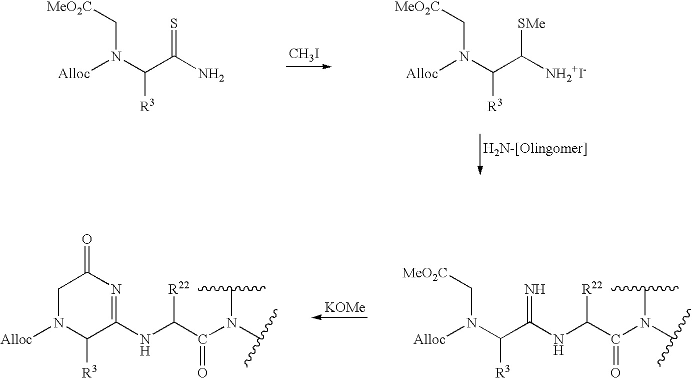

In either scheme, the initial methyl ester can be replaced by a more reactive ester leaving group to further facilitate the cyclization. Additionally in either scheme, the allyloxycarbonyl protecting group can be replaced with other nitrogen-protecting groups, such as benzyloxycarbonyl (Cbz) or fluorenylmethoxycarbonyl (Fmoc).

The dihydropyrazinone peptide analog can be chain-elongated by removal of the nitrogen protecting group and coupling with a nitrogen-protected amino acid derivative under the conventional conditions for this type of reaction, as will be readily apparent to those skilled in the art. This sequence of reactions is shown below:

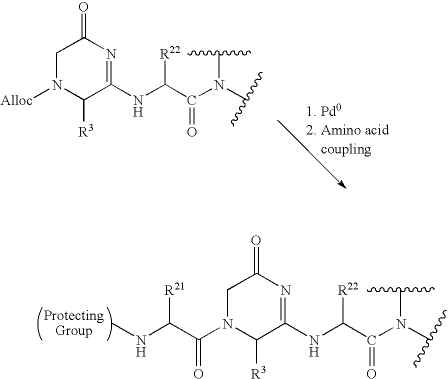

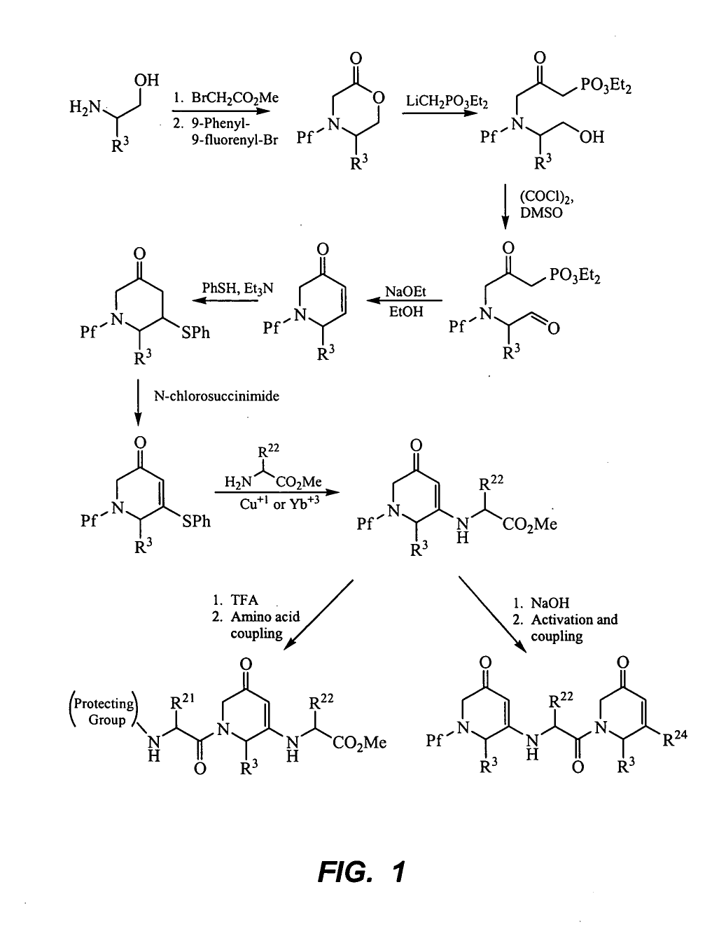

The α-substituted dihydropyridinone (in which R¹ is CH₂ and R² is CH) can be prepared from an amino alcohol precursor, examples of which are commercially available or readily obtained from natural and unnatural amino acids by reduction of the carboxyl group. Alkylation of the amino group with methyl bromoacetate is followed by cyclization to the lactone. The lactone is reacted with 9-phenylfluorenyl bromide to introduce the 9-phenyl-9-fluorenyl (“Pf”) protecting group on the nitrogen; the Pf group is known in the art for its ability to inhibit racemization at the adjacent carbon stereocenter (Lubell, W. D., Rapoport, H., J. Am. Chem. Soc. 1987, vol. 109, 236). Other protecting groups commonly employed in peptide synthesis may also be used. Reaction of the protected lactone with the lithium salt of diethyl methylphosphonate affords the ketophosphonate, the alcohol of which is oxidized to the aldehyde with the Swern reagent (Mancuso, A. J.; Swern, D. Synthesis, 1981, 165-185). The aldehyde is cyclized on treatment with base, as described by Grison, C., et al., Tetrahedron 2001, vol. 57, 4903; Hintermann, T., et al., Helv. Chim. Acta 1998, vol. 81, 983. Conversion of the enone functionality to the β-phenylthioenone is accomplished using the procedure of De Groot, A., et al., Synth. Commun. 1987, vol. 17, 1607. The activated dihydropyridinone is coupled with an amino acid ester with catalysis by Cu⁺¹ or a lanthanide Lewis acid such as Yb⁺³. The coupled product may be further extended at either the N-terminus or C-terminus by selective deprotection and conventional coupling reactions with amino acid derivatives or with azacyclohexenone derivatives. An illustrative reaction scheme is shown in FIG. 1.

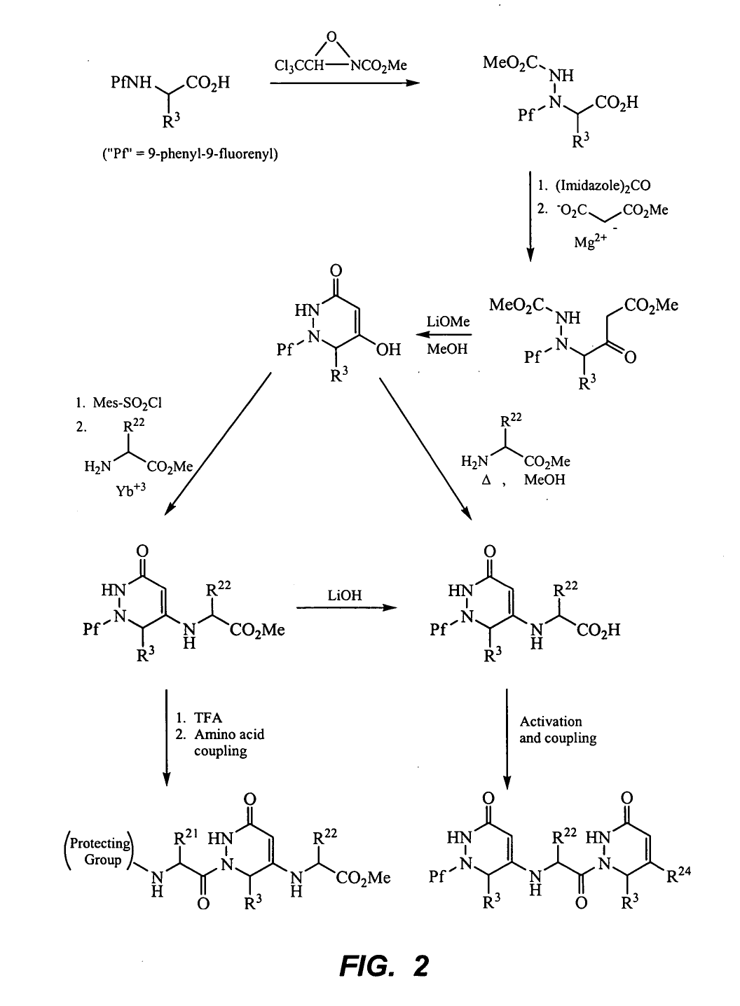

The dihydropyridazinone (in which R¹ is NH and R² is CH) can be prepared from a natural or unnatural amino acid. The nitrogen-protected form is aminated by the method of Vidal, J., et al., Tetrahedron Lett. 1998, vol. 39, 8845, and the carbonyl group is converted to the β-ketoester using the method of Brooks, D. E., et al., Angew. Chem. Intl. Ed. Engl. 1979, vol. 18, 72. Cyclization is induced by base and the carbamate protecting group is either cleaved under these conditions or removed in a separate step. The dihydropyridazinone may be coupled directly with an amino acid by heating in an alcoholic solvent, or it may be activated by reaction with, for example, mesitylenesulfonyl chloride, followed by coupling with an amino acid ester with catalysis by a lanthanide Lewis acid such as Yb⁺³. The coupled product may be further extended at either the N-terminus or C-terminus by selective deprotection and conventional coupling reactions with amino acid derivatives or with azacyclohexenone derivatives. An illustrative reaction scheme is shown in FIG. 2.

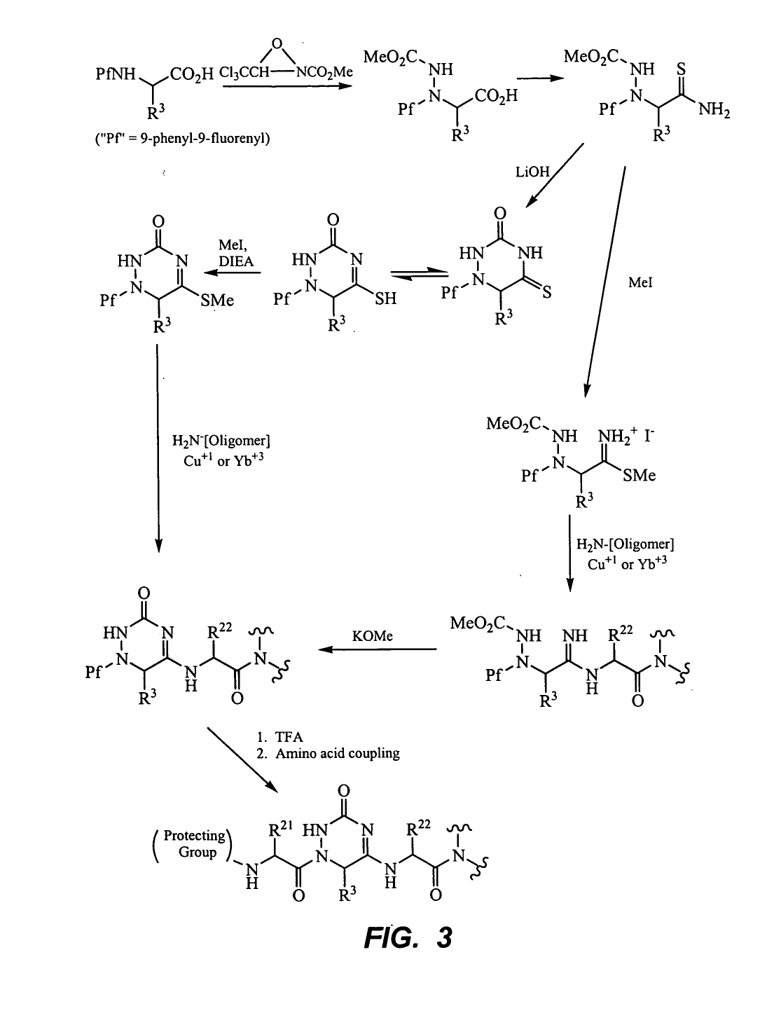

The dihydrotriazinone (in which R¹ is NH and R² is N) can be prepared from a natural or unnatural amino acid. The amino acid is first aminated as illustrated in the synthesis of the dihydropyridazinone. The carboxyl group is then converted to the thionoamide, which is cyclized with base. The thioimide is alkylated with methyl iodide and then coupled to an amino acid derivative or a peptide derivative with catalysis by Cu(I) or a soft Lewis. Alternatively, the thioamide may be alkylated with methyl iodide and the thioamidate salt can be converted to the amidine under neutral conditions. The amidine can then be cyclized at the less hindered nitrogen to give the triazinone. An illustrative reaction scheme is shown in FIG. 3.

Each of the products of the reactions shown in FIGS. 1, 2, and 3 can be N-deprotected and coupled to another amino acid using conventional procedures. Alternatively, the azacyclohexenone-amino acid ester derivatives depicted in FIGS. 1 and 2 can be deprotected at the carboxyl group and the resulting acid then coupled as a unit for more rapid chain elongation.

The α-substituted azacyclohexenone enantiomers that correspond to the L configuration of the natural amino acids can be obtained through synthesis, beginning with an amino acid or amino acid derivative of the natural configuration as starting material, as outlined in the Examples below. Alternatively, if synthetic intermediates are produced in racemic form, they may be resolved into the separate enantiomers prior to incorporation in the peptide analog. The desired stereoisomer may also be obtained from peptide analogs in which both enantiomers of an α-substituted azacyclohexenone are present as a mixture, using chromatography or other means known in the art.

Coupling can also be performed by solid phase synthesis. For example, an Fmoc-protected amino acid coupled to a solid resin such as a Wang or Merrifield polystyrene resin can be deprotected with 20% piperidine in DMF, then coupled to an activated and N-protected form of the azacyclohexenone in a mixed solvent of methylene chloride and DMF (1:3.5 volume ratio), followed by treatment with acetic anhydride, DIEA, and methylene chloride (1:1:3). The N-protecting group is then removed, and the steps repeated until the desired peptide analog chain is achieved.

Constructs consisting of an all-amino-acid segment linked to a segment in which one or more (but not all) amino acids are replaced by an azacyclohexenone group are readily synthesized by methods analogous to those described above, with the β-turn-promoting linkage added at the appropriate site. Solid phase synthesis is readily used, and the azacyclohexenone units can be incorporated at either the N-terminus or the C-terminus of the hybrid. C-Terminal azacyclohexenone incorporation, for example, can be performed by solid-phase synthesis of the desired azacyclohexenone-containing segment, followed by coupling to amino acids using standard peptide coupling reactions. N-terminal azacyclohexenone incorporation can be performed by first synthesizing the solid phase-bound peptide segment, followed by coupling to the azacyclohexenone-containing segment.

Formulas and Administration of the Peptide Analogs and Constructs of the Invention

When used as drugs for administration to mammals, the compounds of this invention can be administered in water-soluble form and preferably as pharmaceutically acceptable salts. Pharmaceutically acceptable salts are those that retain the biological effectiveness of the free bases or acids without introducing unfavorable side effects. The salts can be either acid or base addition salts, depending on the peptide analog itself. Examples of acceptable acid addition salts are those formed with inorganic acids such as hydrochloric, hydrobromic, sulfuric, nitric, or phosphoric acid, and those formed with organic acids such as acetic, propionic, glycolic, pyruvic, oxalic, maleic, malonic, succinic, fumaric, tartaric, citric, benzoic, cinnamic, mandelic, methanesulfonic, ethanesulfonic, p-toluenesulfonic, or salicylic acid. Examples of acceptable base addition salts are those formed with inorganic bases such as sodium, potassium, lithium, ammonium, calcium, magnesium, iron, zinc, manganese, or aluminum hydroxide, and those formed with organic bases such as primary, secondary, and tertiary amines such as isopropylamine, trimethylamine, diethylamine, triethylamine, tripropylamine, and ethanolamine, or with basic ion exchange resins.

The compounds can be formulated into suitable pharmaceutical preparations for administration by intravenous injection, intramuscular injection, intravenous infusion, oral administration, or any other conventional methods of administration. The active ingredient can be compounded, for example, with non-toxic, pharmaceutically acceptable carriers and excipients that are common to pharmaceutical formulations in general, as aqueous solutions, or as emulsions or suspensions, or in solid or semi-solid forms such as tablets, pellets, capsules, or suppositories. Examples of suitable carriers are water, glucose, lactose, gum acacia, gelatin, mannitol, starch paste, magnesium trisilicate, talc, corn starch, keratin, colloidal silica, potato starch, urea. Excipients can include agents for stabilization, thickening, coloring, or scent, or agents to aid in formulating the dosage forms, selected as needed in accordance with the intended manner of administration as well as the particular condition to be treated. Tablets for oral administration, for example, can contain microcrystalline cellulose, sodium citrate, calcium carbonate, dicalcium phosphate, or glycine, along with any of various disintegrants such as corn, potato, or tapioca starch, alginic acid or complex silicates, together with granulation binders such as polyvinylpyrrolidone, sucrose, gelatin or acacia. Lubricating agents such as magnesium stearate, sodium lauryl sulfate or talc can also be included.

The amount of active ingredient to be included in a single dosage form will vary depending on the patient to be treated and the particular mode of administration. The optimal dose level for a particular patient will depend on such factors as the age, body weight, general health, sex, and diet of the patient, as well as the time of administration, the route of administration, the rate of excretion, the severity of the disease being treated, and whether or not the patient is simultaneously undergoing any other drug therapy. In most cases, the amount of active ingredient administered will range from about 1 to about 1,000 mg per day, preferably from about 10 to about 500 mg per day.

The following examples are offered for purposes of illustration, and are not intended to impose limits on the scope of the invention.

Reagents and solvents were obtained from commercial suppliers and were used as received unless otherwise noted. Dichloromethane, MeCN, TEA, and DIEA were distilled from calcium hydride, THF was distilled from Na. Solvents were removed with a rotary evaporator at aspirator pressure, followed by drying under high vacuum. Flash chromatography was performed according to the method described by Still et al. (Still, W. C.; Kahn, M.; Mitra, A. J. Org. Chem. 1978, 43, 2923) using 60 Å pore, 200-mesh silica gel from E. Merck & Co. and the solvent system indicated. Analytical thin-layer chromatography (TLC) was performed using Merck silica gel 60 F₂₅₄ plates.

Analytical high pressure liquid chromatography (HPLC) analysis was performed using a Waters 996 Photodiode Array Detector with a Waters 600 Controller and Pump. The analytical column was a Varian DYNAMAX-100 Å (4.6 mm×250 mm) reversed-phase C₁₈ column. Retention times were recorded using the gradient method A (flow rate 1 mL/min). Chiral analytical HPLC was performed using an Hewlett Packard Series 1100 Quatpump and MWD. The analytical column used was a Chiralcel OD normal phase column. Retention times were recorded using a solvent system of hexanes/isopropanol 85:15. Preparative HPLC purification was performed using a Waters 486 Tunable Absorbance Detector and a Waters 600 Controller and Pump. The column was a Varian DYNAMAX-100 Å (21.4 mm×250 mm) reversed-phase C₁₈ column equipped with a guard column. For purification purposes, the gradient program used was Method B (flow rate 15 mL/min). Liquid Chromatography-Mass Spectometry (LCMS) analysis was performed using an HP Series 1100 Quatpump, MWD, and MSD. The analytical column used was a Zorbax SB-C18 reverse-phase column (2.1 mm×5 cm). The gradient method C was used for analysis (flow rate 0.4 mL/min).

| HPLC Method A | | | | LCMS Method | |
|---|---|---|---|---|---|
| | % CH₃CN | HPLC Method B | | | % CH₃CN |
| Time | (+0.1% TFA) | Time | % CH₃CN | Time | (+0.1% TFA) |
| 0 | 10 | 0 | 0 | 0 | 5 |
| 0-25 | Ramp to 95 | 0-40 | Ramp to 30 | 0-8 | Ramp to 95 |
| 30 | 95 | 40-45 | Ramp to 95 | 8-10 | 95 |
| 32 | Ramp to 10 | 50 | 95 | 10-12 | Ramp to 5 |
| | | 55 | Ramp to 0 | 12-14 | 5 |

Infrared (IR) spectra were recorded with a Perkin-Elmer 1600 Fourier transform infrared spectrometer and are reported in wavenumbers (cm⁻¹). Nuclear Magnetic Resonance (NMR) spectra were obtained using Bruker 300, 400, or 500 MHz spectrometers with ¹³C operating frequencies of 100 and 125 MHz. Spectral data are reported as chemical shifts (multiplicity, number of hydrogens, coupling constants in Hz). Chemical shifts were calibrated to the solvent peak (3.31 for CD₃OD, 7.27 ppm for CDCl₃, 2.50 for (CD₃)₂SO). Peaks for the major rotamer are reported, and representative peaks of the minor rotamer are also reported. All ¹³C NMR were proton decoupled and referenced to CDCl₃ (77.0 ppm) or (CD₃)₂SO (39.5 ppm).

### EXAMPLE 1

This example illustrates the liquid-phase synthesis of a two-unit construct of the present invention that includes an N-protected dihydropyrazinone of the present invention linked to a carboxy-protected valine residue. The construct is (6RS)-6-isobutyl-5-((1S)-1-t-butoxycarbonyl-2-methylpropylamino)-3-oxo-3,6-dihydro-2H-pyrazine-1-carboxylic acid allyl ester, or by an abbreviated name Alloc-[Leu]-Val t-butyl ester in which the brackets denote the dihydropyrazinone counterpart of the amino acid (in this case, leucine), and its formula is as follows:

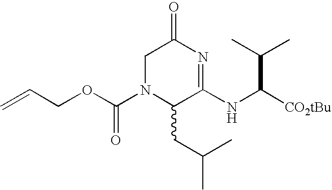

Sections A through F below illustrate one reaction scheme to this construct.

### 1.A. N-(2-Methoxy-2-oxoethyl)-L-Leucine Amide (2) from L-Leucine Amide Hydrochloride (1)

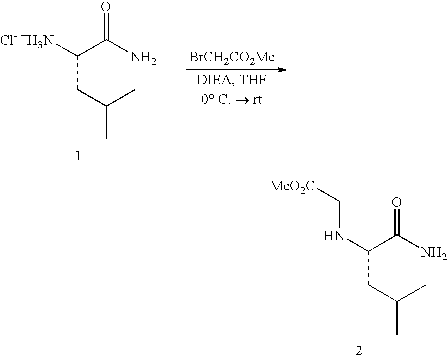

To 2.0 g (12 mmol) of L-leucine amide hydrochloride (1) in 20 mL of dry THF at 0° C. was added 5.0 mL (29 mmol) of DIEA followed by slow addition of a solution of methyl bromoacetate (1.4 mL, 14 mmol) in dry THF (20 mL). The reaction solution was stirred under argon and allowed to warm up to room temperature over a 20 h period before it was diluted with 50 mL of saturated NH₄Cl. After concentration of the reaction mixture, the product was extracted with EtOAc (2×50 mL). The organic layer was further washed with saturated NaHCO₃ (50 mL) and brine (50 mL), dried over Na₂SO₄, filtered, and concentrated. The crude product was purified by flash chromatography (DCM/MeOH/TEA 99:1:1→95:5:1) to afford 2 as a yellow oil (1.1 g, 47% yield). If the column chromatography is performed without TEA, the product is isolated as a white solid. R_{f}=0.26 (KMnO₄/basic, DCM/MeOH 95:5); retention time (analytical HPLC)=13.1 min; IR (film) 1673, 1745, 2955 cm¹; ¹H NMR (400 MHz, CDCl₃) δ 0.90 (d, 3, J=4.7), 0.92 (d, 3, J=4.7), 1.44 (m, 1, J=5.5, 8.8, 14), 1.54 (m, 1, J=4.8, 8.8, 14), 1.73 (m, 1), 2.1 (s, 1), 3.08 (dd, 1, J=4.8, 8.8), 3.29 (d, 1, J=17.5), 3.43 (d, 1, J=17.5), 3.68 (s, 3), 6.42 (s, 1), 7.00 (s, 1); ¹³C NMR (125 MHz, CDCl₃) δ 21.8, 23.2, 24.9, 43.1, 49.2, 51.9, 61.2, 172.6, 178.1; MS (ES) m/z 203.1 (M+H⁺), 158.1 (M+H⁺-CONH₂); HRMS (FAB) m/z 203.1398 (M+H⁺ C₉H₂₀N₂O₃ requires 203.1396).

### 1.B. N-Allyloxycarbonyl-N-(2-methoxy-2-oxoethyl)-L-Leucine Amide (3) From N-(2-Meth0xy-2-oxoethyl)-L-Leucine Amide (2)

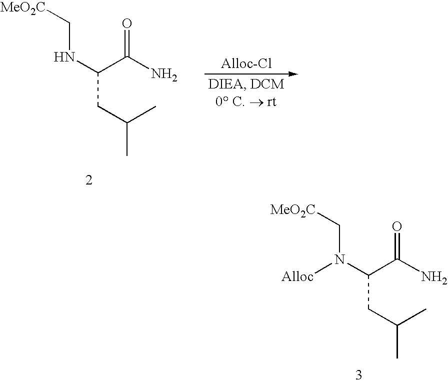

To 0.37 g (1.8 mmol) of 2 in 18 mL of dry DCM at 0° C. was added 2.0 mL (11.1 mmol) of DIEA followed by slow addition of allyl chloroformate (0.55 mL, 5.2 mmol). The reaction solution was stirred under argon and allowed to warm up to room temperature over a 20 h period before it was diluted with 50 mL of saturated NH₄Cl. After concentration of the reaction mixture, the product was extracted with EtOAc (3×10 mL). The organic layer was further washed with saturated NH₄Cl (2×15 mL), saturated NaHCO₃ (15 mL), and brine (15 mL), dried over Na₂SO₄, filtered, and concentrated. The crude product was purified by flash chromatography (EtOAc/hexanes 1:2→1:1→2:1) to afford 3 as a yellow oil (0.41 g, 79% yield). R_{f}=0.51 (ninhydrin, 12, EtOAc/hexanes 10:3); ¹H NMR (500 MHz, CDCl₃, rotamers) δ 0.92-0.99 (m, 6), 1.50-1.59 (m, 1), 1.59-1.66 (m, 1, J=4.0), 1.79-1.85 (m, 0.6, J=5.5), 1.90-1.96 (m, 0.5, J=4.5), 3.74 (s, 1.6), 3.79 (s, 1.4), 3.95-4.06 (m, 2), 4.42 (m, 0.4), 4.59 (dt, 1, J=1.5, 5.5), 4.63 (dd, 1, J=4.2, 5.8), 4.71 (dd, 0.7, J=6.0, 9.0), 5.19-5.35 (m, 2, J=1.5) 5.84-5.90 (m, 1), 6.44 (s, 0.6), 6.54 (s, 0.4), 7.28 (s, 0.6), 7.78 (s, 0.4); ¹³C NMR (125 MHz, CDCl₃, rotamers) δ 21.3/21.7, 23.0/23.2, 24.7/24.7, 37.5/38.2, 45.9/#, 52.4/52.7, 57.7/59.3, 66.7/67.0, 117.6/118.4, 131.8/132.1, 155.8/155.8, 171.0/171.8, 173.2/173.6; MS (ES) m/z 309.2 (M+Na⁺), 242.1 (M+H⁺-CONH₂); HRMS (FAB) m/z 287.1611 (M+H⁺ C₁₃H₂₃N₂O₅ requires 287.1607).

### 1.C. N-Allyloxycarbonyl-N-(2-methoxy-2-oxoethyl)-L-Leucine Thioamide (4) From N-Allyloxcarbonyl-N-(2-methoxy-2-oxoethyl)-L-Leucine Amide (3)

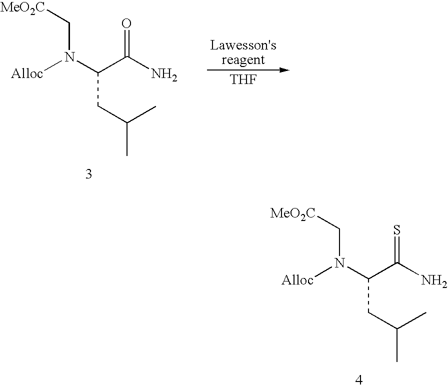

To 1.0 g (3.5 mmol) of N-allyloxycarbonyl-N-(2-methoxy-2-oxoethyl)-L-leucine amide 3 in 50 mL of dry THF was added 0.99 g (2.4 mmol) of Lawesson's reagent, which turns the solution faint yellow and opaque. The reaction solution was stirred under argon at room temperature, and the reaction was stopped at 1.5 h when no more starting material was observed by TLC. After concentration of the reaction mixture, the crude product was redissolved in EtOAc. The organic layer was washed with saturated NaHCO₃ (2×50 mL). The basic aqueous layer was acidified by addition of solid KHSO₄ and extracted with EtOAc (2×20 mL). The combined organic layers were further washed with brine (50 mL), dried over Na₂SO₄, filtered, and concentrated. The crude product was purified by flash chromatography (EtOAc/hexanes 1:2→1:1) to afford 4 as a white solid (0.82 g, 78% yield). R_{f}=0.55 (UV, EtOAc/hexanes 1:1); retention time (analytical HPLC): 16.2 min; retention time (chiral HPLC): 7.1 min; ¹H NMR (500 MHz, CDCl₃, rotamers) δ 0.94-0.96 (m, 6), 1.46-1.56 (m, 1), 1.64-1.72 (m, 1), 2.22 (m, 0.5, J=6.0, 8.6, 14.5), 2.47 (m, 0.5, J=4.0, 10.7, 14.8), 3.75 (s, 1.5), 3.82 (s, 1.5), 3.83 (m, 1), 4.09 (t, 1, J=18.0), 4.61 (dd, 1, J=1.4, 5.4), 4.65 (t, 1, J=5.4), 4.86 (dd, 0.4, J=3.6, 11.1), 4.93 (dd, 0.6, J=6.1, 9.2), 5.21-5.35 (m, 2, J=1.3), 5.82-5.92 (m, 1), 7.60 (s, 0.5), 7.68 (s, 0.5), 8.66 (s, 0.5), 9.53 (s, 0.5); ¹³C NMR (125 MHz, CDCl₃, rotamers) δ 20.9/21.8, 22.9/23.4, 25.0/25.2, 40.3/41.4, 45.6/46.3, 52.5/53.0, 62.8/64.9, 67.0/67.3, 117.8/118.8, 131.6/132.0, 156.0/156.4, 171.1/172.5, 207.0/207.7; MS (ES) m/z 303.1 (M+H⁺); HRMS (FAB) m/z 303.1373 (M+H⁺ C₁₃H₂₃N₂O₄S requires 303.1378).

### 1.D. (2S)-2-Isobutyl-5-oxo-3-thioxopiperazine-1-carboxylic Acid Allyl Ester (5) From N-Allyloxycarbonyl-N-(2-methoxy-2-oxoethyl)-L-Leucine Thioamide (4)

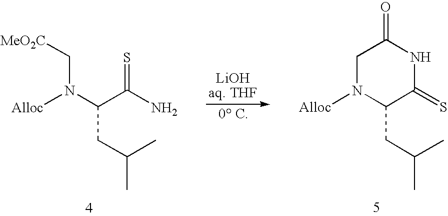

To 0.24 g (0.78 mmol) of 4 in 16 mL of THF/H₂O 3:1 at 0° C. was added 1 M LiOH (0.78 mL, 0.78 mmol) dropwise. The reaction solution was stirred under nitrogen and quenched after 6 min by addition of solid KHSO₄. After concentration of the reaction mixture, the aqueous solution was extracted with EtOAc (2×10 mL). The organic layer was further washed with 1 M KHSO₄ (3×10 mL), saturated NaHCO₃ (3×10 mL), and brine (10 mL), dried over Na₂SO₄, filtered, and concentrated. No further purification was necessary to afford 5 as a yellow oil (0.15 g, 71% yield). R_{f}=0.6 (UV, EtOAc/hexanes 1:1); retention time (analytical HPLC): 15.8 min; retention time (chiral HPLC): 8.0 min; ¹H NMR (500 MHz, CDCl₃) δ 0.92 (d, 3, J=6.5), 0.94 (d, 3, J=5.9), 1.58-1.74 (m, 3), 3.6-3.8 (m, 1), 4.58 (m, 2, J=4.8), 4.8-5.0 (m, 1), 5.18 (dd, 1, J=1.1, 10.4), 5.26 (dd, 1, J=1.3, 17.2), 5.2-5.4 (m, 1), 5.8 (m, 1, J=5.6, 10.8), 10.4 (s, 1); ¹³C NMR (125 MHz, CDCl₃) δ 21.0, 22.8, 24.6, 41.2, 42.2, 61.1, 67.0, 118.5, 131.6, 154.0, 165.1, 206.5; MS (ES) m/z 271.1 (M+H⁺).

### 1.E. (6S)-6-Isobutyl-5methylsulfanyl-3-oxo-3,6-dihydro-2H-pyrazine-1-carboxylic Acid Allyl Ester (6) From (2S)-2-Isobutyl-5-oxo-3-thioxopiperazine-1-carboxylic Acid Allyl Ester (5)

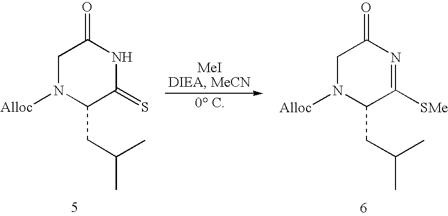

To 0.11 g (0.4 mmol) of 5 in 15 mL of dry MeCN at 0° C. was added 25 μL (0.4 mmol) of methyl iodide followed by slow addition of a solution of DIEA (80 μL, 0.46 mmol) in 10 mL MeCN via an addition funnel over the course of 30 min while the reaction solution was stirred under argon. After all of the base was added, the flask was capped and the solution was stirred for an additional 2 h. After concentration of the reaction mixture, the crude product was redissolved in EtOAc. The organic layer was washed with NaH₂PO₄ (2×20 mL) and brine (20 mL), dried over Na₂SO₄, filtered, and concentrated. The crude product was used without further purification. R_{f}=0.52 (UV, EtOAc/hexanes 1:1); ¹H NMR (400 MHz, CDCl₃) δ 0.98 (m, 6, J=4.4), 1.4-1.5 (m, 1), 1.6-1.7 (m, 2), 2.51 (s, 3), 3.9-4.0 (m, 1), 4.58-4.63 (m, 1), 4.62 (d, 2, J=4.8), 4.73-4.74 (m, 0.5), 4.85-4.95 (m, 0.5), 5.26 (d, 1, J=10.4), 5.32 (d, 1, J=17.2), 5.92 (m, 1, J=5.6, 10.8); MS (ES) m/z 285.1 (M+H⁺).

### 1.F. (6S)-6Isobutyl-5-((1S)-1-t-butoxycarbonyl-2-methylpropylamino)-3-oxo-3,6-dihydro-2H-pyrazine-1-carboxylic acid Allyl Ester (7) From (6S)-6Isobutyl-5-methylsulfanyl-3-oxo-3,6-dihydro-2H-pyrazine-1-carboxylic Acid Allyl Ester (6)

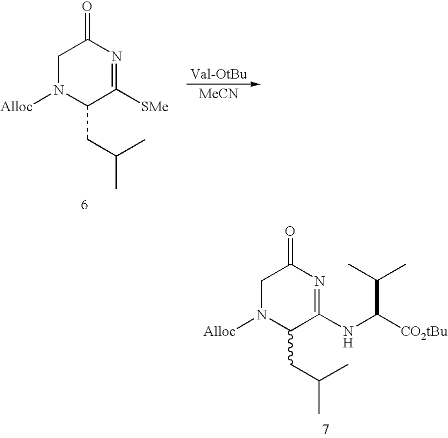

To a solution of crude acyl thioimidate 6 (97.4 mg, 0.34 mmol) in 5 mL of dry MeCN was added 1.1 equivalents of valine t-butyl ester, and the reaction solution was allowed to stir under argon overnight. After concentration of the reaction mixture, the crude product was purified by flash chromatography (DCM/MeOH 99:1) to afford 7 as a yellow oil (36% yield over two steps, 5/1 ratio of S/R stereoisomers of the dihydropyrazinone unit).

### EXAMPLE 2

This example illustrates an alternative synthetic route to the construct prepared in Example 1. This route begins with Sections 1.A through 1.C of Example 1 and continues with Sections 2.D through 2.F below.

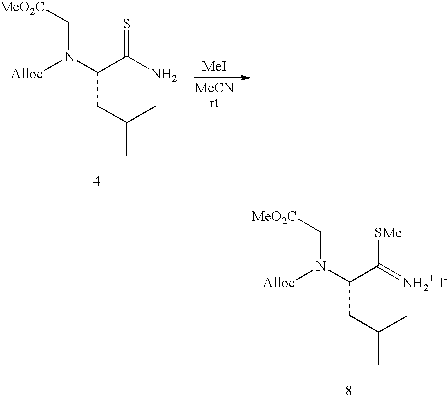

To 0.05 g (0.16 mmol) of 4 in 5 mL of dry MeCN was added 31 μL of (0.50 mmol) methyl iodide and the reaction solution was stirred under argon at room temperature for 4 h. After concentration of the reaction mixture, the crude product (8) was used without further purification. ¹H NMR (500 MHz, CDCl₃, crude, rotamers/diastereomers) δ 0.86-0.93 (m, 6), 1.48-1.56 (m, 1), 1.73 (m, 1, J=4.6, 9.4, 14.0), 2.05 (m, 0.3, J=3.6, 10.6, 14.3), 2.13 (m, 0.7, J=3.9, 10.4, 14.4), 2.92 (s, 2), 2.94 (s, 1), 3.74 (s, 3), 4.15-4.29 (m, 2), 4.50 (d, 1.3, J=5.5), 4.56 (d, 0.7, J=6.5), 5.14-5.23 (m, 3), 5.75 (m, 0.7, J=5.4, 10.7, 16.1), 5.8-5.85 (m, 0.3); MS (ES) m/z 317.3 (M+H⁺).

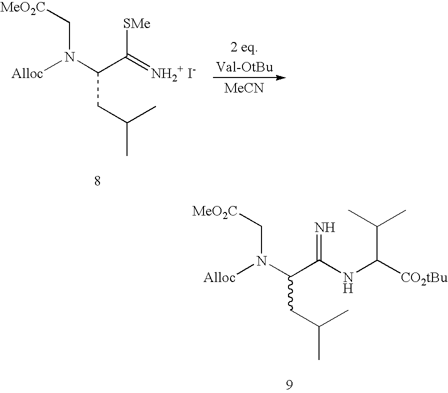

To a crude product mixture containing 44.8 mg (01.4 mmol) of 8 in 1 mL of dry DCM was added 1.1 equivalents of valine t-butyl ester, and the reaction solution was stirred under argon for 22 h. After concentration of the reaction mixture, the crude product was purified by flash chromatography (EtOAc/hexanes 1:1→EtOAc→MeOH/EtOAc 1:9) to afford 9 as a yellow oil (46.7 mg, 75% yield over two steps). R_{f}=0.13 (UV, EtOAc/hexanes 1:1); ¹H NMR (500 MHz, (CD₃)₂SO) δ 0.85-1.0 (m, 12), 1.44 (s, 9), 1.50-1.60 (m, 1), 1.55-1.75 (m, 1), 1.85-1.95 (m, 1), 2.20 (m, 1), 3.70 (s, 3), 4.15-4.40 (m, 3), 4.55 (s, 2), 4.75-4.90 (m, 1), 5.17-5.25 (m, 2), 5.80-5.95 (m, 1), 9.17 (s, 1), 9.48 (s, 1); MS (ES) m/z 442.4 (M+H⁺).

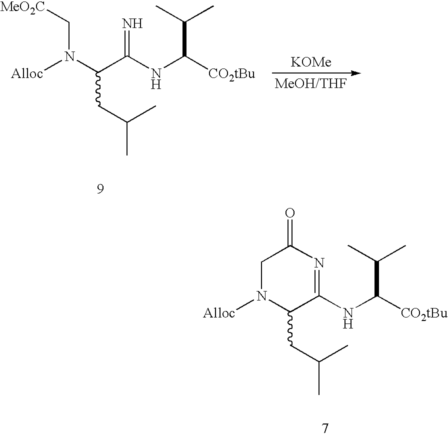

To a solution of 1.09 g (2.5 mmol) of the amidine 9 in 150 mL of 5:1 THF/MeOH at 0° C. was added 0.2 g (2.8 mmol) of potassium methoxide. The reaction solution was allowed to stir under nitrogen and then neutralized after 20 min by addition of 1 M KHSO₄. The organic layer was washed with KHSO₄ (50 mL). The acidic aqueous layer was extracted with EtOAc (2×50 mL). The combined organic layers were washed with NaCl (1×50 mL), dried over Na₂SO₄, filtered, and concentrated. The crude product 7 was used without further purification (0.71 g, 70% yield, 2/1 ratio of S/R stereoisomers of the dihydropyrazinone unit).

### EXAMPLE 3

This example illustrates an alternative synthetic route to the construct prepared in Example 1. This route begins with Sections 1.A through 1.D of Example 1 and continues with Section 3.E below.

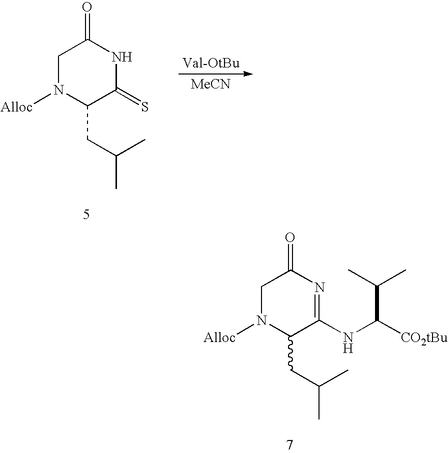

To a solution of thioimide 5 (50 mg, 0.18 mmol) in 5 mL of isopropanol at 50° C. was added valine t-butyl ester (1 equivalent), and the reaction solution was allowed to stir under nitrogen overnight. After concentration of the reaction mixture, the crude product was purified by flash chromatography (EtOAc/hexanes 1:3→1:2→1:1) to afford racemic starting material 5 (27% yield) and 7 as a yellow oil (17% yield, 1/2 ratio of S/R stereoisomers of the dihydropyrazinone). R_{f}=0.39 (UV, EtOAc/hexanes 1:1); IR (film) 1390.6, 1709.8, 2872.8, 2961.1 cm⁻¹; ¹H NMR (500 MHz, (CD₃)₂SO, 30° C., rotamers and diastereomers) δ 0.89-0.97 (m, 12), 1.26-1.30 (m, 1, J=9.7), 1.4 (s, 9), 1.50-1.60 (m, 1), 1.65-1.75 (m, 1, J=10.8), 2.13 (m, 1, J=6.5), 3.68 (d, 0.4, J=18.3), 3.78 (d, 0.6, J=18.0), 4.19 (d, 1, J=18.4), 4.33 (t, 0.25, J=8.1), 4.40 (t, 0.75, J=6.7), 4.57 (s, 2), 5.00-5.10 (m, 1), 5.19-5.28 (m, 2), 5.85-6.00 (m, 1), 8.80 (d, 0.25, J=7.5), 8.98 (d, 0.3, J=7.9), 9.02 (d, 0.45 J=6.8); MS (ES) m/z 410.2 (M+H⁺), 354.3 (M+H+-tBu).

A side product of the reaction of Section 3.E is N-(N-allyloxycarbonyl-N-(3-methyl-1-thiocarbamoylbutyl)-glycyl)-L-valine t-butyl ester, whose structure and confirmatory data are shown below: 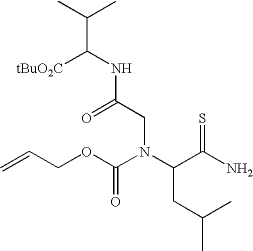
R_{f}=0.55 (UV, EtOAc/hexanes 1:1); ¹H NMR (300 MHz, (CD₃)₂SO, rotamers/diastereomers) δ 0.87-0.93 (m, 12), 1.42 (s, 9), 1.60-1.80 (m, 2), 1.90-2.10 (m, 2), 4.00-4.15 (m, 3), 4.46-4.50 (m, 2), 4.50-4.70 (m, 1), 5.08-5.24 (m, 2), 5.75-5.95 (m, 1), 8.43 (d, 1, J=9.0), 9.72 (s, 0.6), 9.75 (s, 0.4), 10.34 (s, 0.6), 10.52 (s, 0.4); MS (ES) m/z 466.3 (M+H⁺), 388.2 (M+H+-tBu).

### EXAMPLE 4

This example illustrates the deprotection of (6RS)-6-isobutyl-5-((1S)-1-t-butoxycarbonyl-2-methylpropylamino)-3-oxo-3,6-dihydro-2H-pyrazine-1-carboxylic acid allyl ester (7), followed by separation of the product into L,L- and D,L-diastereomers. The L,L-diastereomer is then subjected to a series of amino acid couplings. As noted in Example 1, the dihydropyrazinone unit is denoted by the three-letter code for the amino acid bearing the same side chain, the code being presented in brackets to indicate that it refers to the dihydropyrazinone unit rather than the amino acid. Bracketed three-letter codes that are not preceded by a superscript denote the (S)-stereoisomer of the unit. The dihydropyrazinone unit in this example is thus abbreviated as [Leu]. Bracketed three-letter codes that are preceded by a superscript D, which appear in Example 5, denote the (R)-stereoisomer of the unit.

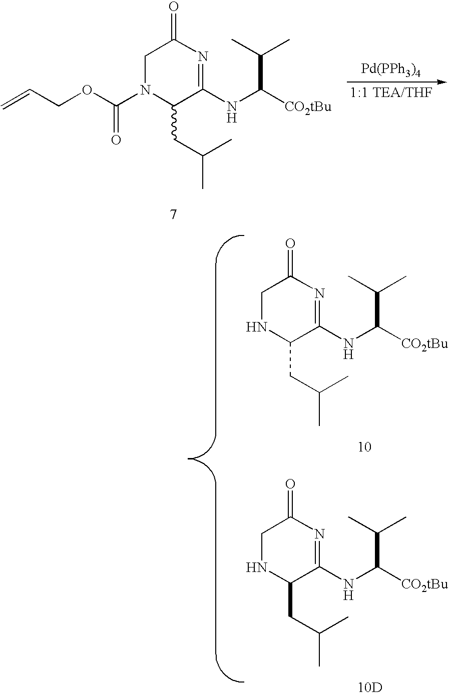

To 0.87 g (2.1 mmol) of 7 in 10 mL dry THF was added 10 mL of diethylamine (97 mmol), followed by Pd(PPh₃)₄ (0.48 g, 0.4 mmol). The reaction solution was stirred under argon at room temperature for 3 h. After concentration of the reaction mixture, the crude product was purified by flash chromatography to separate the diastereomers (1:99:0.54→2:98:0.5→3:97:0.5 EtOH/EtOAc/TEA). The overall yield of the reaction was 69%.

L,L-Diastereomer (10): R_{f}=0.32 (UV, EtOH/EtOAc/TEA 10:90:1); ¹H NMR (400 MHz, CD₃OD) δ 0.98-1.04 (m, 12), 1.39 (m, 1, J=5.0, 9.6, 14.3), 1.49 (s, 9), 1.72 (m, 1, J=4.6, 10.7, 15.3), 1.70-1.80 (m, 1), 2.12 (m, 1, J=6.6), 3.36 (d, J=18.3), 3.44 (d, J=18.3), 3.69 (dd, J=5.0, 10.8), 4.65 (d, J=5.9); MS (ES) m/z 326.3 (M+H⁺), 270.3 (M+H⁺-tBu).

D,L-Diastereomer (10D): R_{f}=0.45 (UV, EtOH/EtOAc/TEA 10:90:1); ¹H NMR (400 MHz, CD₃OD) δ 0.83-0.92 (m, 12), 1.20 (m, 1, J=5.1, 9.2, 14.1), 1.38 (s, 9), 1.63 (m, 1, J=4.6, 10.6, 13.8), 1.75-1.85 (m, 1), 2.12 (m, 1, J=6.7), 3.25 (d, 1, J=18.4), 3.32 (d, 1, J=18.4), 3.61 (dd, 1, J=5.1, 10.8), 4.50(d, 1, J=6.1); MS (ES) m/z 326.3 (M+H⁺), 270.3 (M+H⁺-tBu).

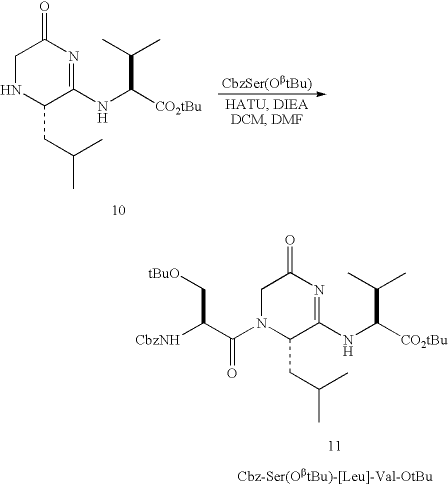

To 125 mg (0.36 mmol) of 10 in 15 mL of dry DCM and 2 mL of dry DMF was added DIEA (1.5 equiv), Cbz-Ser(tBu)-OH (1.5 equiv), and HATU (1.5 equiv). The reaction solution was stirred under argon at room temperature for 5 h. After concentration of the reaction mixture, the crude product was purified by flash chromatography (1:2→1:1 EtOAc/hexanes) to afford 11 as a white solid (75% yield, 9/1 ratio of S/R stereoisomers of the dihydropyrazinone unit). R_{f}=0.43 (UV, EtOAc/hexanes 1:1); retention time (analytical HPLC): 27.0 min; ¹H NMR (500 MHz, (CD₃)₂SO) δ 0.85-0.94 (m, 12), 0.97 (s, 9), 1.30 (m, 1, J=5.6, 10.5, 13.6), 1.40 (s, 9), 1.48-1.56 (m, 1), 1.61 (m, 1, J=5.5, 14.8), 2.13 (m, 1, J=6.6), 3.37-3.44 (m, 2), 3.84 (d, 1, J=17.5), 4.36 (dd, 1, J=5.9, 7.9), 4.46 (d, 1, J=17.6), 4.51 (dd, 1, J=8.0, 14.5), 4.97 (d, 1, J=12.7), 5.07 (d, 1, J=12.7), 5.44 (dd, 1, J=3.8, 11.2), 7.29-7.36 (m, 5), 7.52 (d, 1, J=7.7), 8.86 (d, 1, J=7.9); MS (ES) m/z 603.5 (M+H⁺); HRMS (FAB) m/z 603.3756 (M+H⁺ C₃₂H₅₁N₄O₇ requires 603.3758).

### 4.C. Cbz-Glu(O^{γ}tBu)-Ser(O^{β}tBu)-[Leu]-Val t-Butyl Ester (12) From Cbz-Ser(O^{β}tBu)-[Leu]-Val t-Butyl Ester (11)

To 0.16 g (0.27 mmol) of 11 in 15 mL of methanol under an atmosphere of hydrogen was added 0.03 g (0.03 mmol) of 10% wt Pd/C. The reaction solution was stirred under an atmosphere of hydrogen for 1 h, after which the solution was filtered twice through Celite and concentrated. The crude product was dissolved in 15 mL of dry DCM and 2 mL of dry DMF, and to the reaction solution was added DIEA (1.5 equiv), Cbz-Glu(OtBu)-OH (1.5 equiv), and HATU (1.5 equiv). The reaction solution was stirred under nitrogen at room temperature overnight, then the mixture was concentrated and the crude product was redissolved in EtOAc. The organic layer was washed with NaH₂PO₄ (1×15 mL), saturated NaHCO₃ (1×15 mL), and brine (1×10 mL), dried over Na₂SO₄, filtered, and concentrated. The crude product was purified by flash chromatography (EtOAc/hexanes 1:1→2:1→3:1) to afford 12 as a white solid (74% yield over two steps, 9/1 ratio of S/R stereoisomers of the dihydropyrazinone unit). R_{f}=0.18 (UV, EtOAc/hexanes 1:1); retention time (analytical HPLC): 28.5 min; ¹H NMR (500 MHz, (CD₃)₂SO) δ 0.84-0.94 (m, 12), 0.98 (s, 9), 1.30 (m, 1, J=10.0), 1.37 (s, 9), 1.40 (s, 9), 1.45-1.53 (m, 1), 1.60 (m, 1, J=14.4), 1.65-1.72 (m, 1), 1.74-1.83 (m, 1), 2.14 (m, 1, J=6.5), 2.21 (m, 1), 3.42 (m, 2), 3.84 (d, 1, J=17.5), 4.03 (dd, 1, J=7.8), 4.38 (dd, 1, J=6.2), 4.45 (d, 1, J=17.6), 4.70 (dd, 1, J=7.0), 4.98 (d, 1, J=12.5), 5.02 (d, 1, J=12.6), 5.44 (dd, 1, J=3.2, 11.5), 7.28-7.38 (m, 5), 7.46 (d, 1, J=7.9), 8.23 (d, 1, J=7.3), 8.88 (d, 1, J=7.9); ¹³C NMR (125 MHz, (CD₃)₂SO) δ 18.6, 19.2, 22.0, 24.7, 27.2, 28.0, 28.1, 30.1, 31.6, 45.3, 46.8, 49.3, 53.8, 59.3, 62.3, 65.8, 73.3, 79.9, 81.5, 128.1, 128.2, 128.7, 137.4, 156.2, 169.7, 170.1, 171.5, 172.1, 173.1, 175.9; MS (ES) m/z 788.5 (M+H⁺); HRMS (FAB) m/z 788.4795 (M+H⁺ C₄₁H₆₆N₅O₁₀ requires 788.4810).

### 4.D. Cbz-Lys(N^{ε}Boc)-Glu(O^{γ}tBu)-Ser(O^{β}tBu)-[Leu]-Val t-Butyl Ester (13) From Cbz-Glu(O^{γ}tBu)-Ser(O^{β}tBu)-[Leu]-Val t-Butyl Ester (12)

To 0.14 g (0.18 mmol) of 12 in 15 mL of methanol under an atmosphere of hydrogen was added 0.02 g (0.02 mmol) of 10% wt Pd/C. The reaction solution was stirred under an atmosphere of hydrogen for 3 h, after which the solution was filtered through Celite and concentrated. The crude product was dissolved in 15 mL of dry DCM and 2 mL of dry DMF, and to the reaction solution was added DIEA (1.5 equiv), Cbz-Glu(OtBu)-OH (1.5 equiv), and HATU (1.5 equiv). The reaction solution was stirred under nitrogen at room temperature overnight, then the mixture was concentrated and the crude product was redissolved in EtOAc. The organic layer was washed with NaH₂PO₄ (1×15 mL), saturated NaHCO₃ (1×15 mL), and brine (1×10 mL), dried over Na₂SO₄, filtered, and concentrated. The crude product was purified by flash chromatography (EtOAc/hexanes 2:1→3:1→4:1) to afford 13 as a white solid (86% yield over two steps, 9/1 ratio of S/R stereoisomers of the dihydropyrazinone unit). R_{f}=0.10 (UV, EtOAc/hexanes 1:1); retention time (analytical HPLC): 29.4 min; ¹H NMR (500 MHz, (CD₃)₂SO, major rotamer 10:1) δ 0.84-0.85 (d, 3, J=6.6), 0.89 (d, 3, J=6.4), 0.90 (d, 3, J=6.8), 0.94 (d, 3, J=6.9), 0.98 (s, 9), 1.25-1.35 (m, 4), 1.36 (s, 9), 1.38 (s, 9), 1.41 (s, 9), 1.44-1.52 (m, 2), 1.52-1.64 (m, 2, J=4.0), 1.64-1.72 (m, 1), 1.75-1.84 (m, 1), 2.14 (m, 1, J=6.6), 2.19 (t, 2, J=8.4), 2.87 (m, 2, J=6.2), 3.37-3.44 (m, 2), 3.84 (d, 1, J=17.5), 3.94-3.99 (m, 1), 4.30 (dd, 1, J=7.5, 13.6), 4.38 (dd, 1, J=5.9, 7.9), 4.63 (d, 1, J=17.7), 4.71 (dd, 1, J=8.0), 5.01 (s, 2), 5.45 (dd, 1, J=3.7, 11.6), 6.76 (dd, 1, J=4.9, 6.0), 7.29-7.38 (m, 5), 7.40 (d, 1, J=8.1), 7.95 (d, 1, J=7.9), 8.29 (d, 1, J=7.4), 8.88 (d, 1, J=8.0); (minor rotamer, representative peaks) δ 7.01 (d, 1, J=6.6), 8.02 (d, 1, J=7.9), 8.08 (d, 1, J=8.2), 9.11 (d, 1, J=8.2); ¹³C NMR (125 MHz, (CD₃)₂SO) δ 18.2, 18.9, 21.6, 22.9, 23.3, 24.3, 26.8, 27.6, 27.7, 28.3, 44.9, 46.4, 48.8, 51.1, 54.6, 58.9, 61.9, 65.4, 68.8, 73.0, 77.4, 79.6, 81.1, 127.7, 127.8, 128.4, 137.0, 155.6, 156.0, 169.3, 169.7, 170.7, 171.7, 171.9, 172.7, 174.5, 175.5, 183.7; MS (ES) m/z 1016.8 (M+H⁺); HRMS (FAB) m/z 1016.6304 (M+H⁺ C₅₂H₈₆N₇O₁₃ requires 1016.6284).

### 4.E. Ac-Lys(N^{ε}Boc)-Glu(O^{γ}tBu)-Ser(O^{β}tBu)-[Leu]-Val t-Butyl Ester (14) From Cbz-Lys(N^{ε}Boc)-Glu(O^{γ}tBu)-Ser(O^{β}Bu)-[Leu]-Val t-Butyl Ester (13)

To 60 mg (0.06 mmol) of 13 in 5 mL of methanol under an atmosphere of hydrogen was added 8 mg (0.007 mmol) of 10% wt Pd/C. The reaction solution was stirred under an atmosphere of hydrogen for 2 h, after which the solution was filtered through Celite and concentrated. The crude product was dissolved in 5 mL of dry DCM, and to the reaction solution was added DIEA (100 equiv) and acetic anhydride (100 equiv). The reaction solution was stirred under nitrogen at room temperature for 2 h, then the mixture was concentrated and the crude product was redissolved in DCM. The organic layer was washed with saturated NaHCO₃ (1×10 mL) and brine (1×10 mL), dried over Na₂SO₄, filtered, and concentrated. The crude product 14 was clean except for contamination by DIEA and was used without further purification (quantitative yield over two steps, 9/1 ratio of S/R stereoisomers of the dihydropyrazinone unit). R_{f}=0.44 (UV, EtOH/EtOAc/TEA 10:90:1); retention time (analytical HPLC): 26.3 min; ¹H NMR (500 MHz, (CD₃)₂SO) δ 0.85 (d, 3, J=6.6), 0.89 (d, 3, J=6.4), 0.90 (d, 3, J=6.8), 0.94 (d, 3, J=6.8), 0.98 (s, 9), 1.18-1.52 (m, 33), 1.52-1.72 (m, 4), 1.77-1.84 (m, 1), 1.83 (s, 3), 2.10-2.19 (m, 3), 2.83-2.90 (m, 2), 3.36-3.42 (m, 2), 3.84 (d, 1, J=17.5), 4.19 (dd, 1, J=8.4, 13.5), 4.27 (dd, 1, J=7.6, 13.6), 4.37 (dd, 1, J=5.8, 8.0), 4.45 (d, 1, J=17.7), 4.70 (dd, 1, J=8.2, 14.0), 5.45 (dd, 1, J=4.1, 11.6), 6.73 (t, 1, J=5.2), 7.94 (d, 1, J=5.8), 7.96 (d, 1, J=7.6), 8.17 (d, 1, J=7.5), 8.88 (d, 1, J=7.9); ¹³C NMR (125 MHz, (CD₃)₂SO) δ 12.3, 16.7, 18.0, 18.9, 21.1, 21.6, 22.6, 23.4, 23.7, 24.3, 26.6, 27.7, 27.8, 28.3, 29.8, 31.1, 41.7, 48.9, 51.2, 52.6, 53.4, 55.0, 59.0, 73.0, 77.4, 79.7, 81.2, 153.8, 155.6, 169.3, 169.4, 169.7, 170.7, 171.8, 171.9, 172.1, 172.7, 175.6; MS (ES) m/z 924.8 (M+H⁺); HRMS (FAB) m/z 924.6048 (M+H⁺ C₄₆H₈₂N₇O₁₂ requires 924.6021).

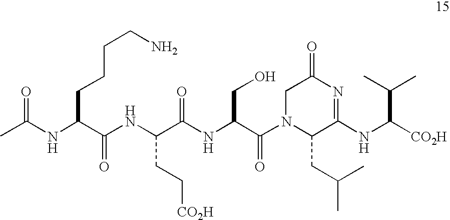

To 24 mg (0.03 mmol) of 14 was added 2.5 mL of trifluoroacetic acid and 1 drop of acetic acid. The reaction solution was warmed and stirred under nitrogen for 6 h. Most of the trifluoroacetic acid was removed by evaporation from dichloroethane (3×10 mL), then saturated NaHCO₃ was added to dissolve the crude product in a basic aqueous solution. The crude sample was filtered before purification by preparatory HPLC (Method B). HPLC fractions containing the pure product were frozen at −78° C. before lyophilization, to yield 15 as a white solid (66% yield, single (S) diastereomer of the dihydropyrazinone unit). Retention time (analytical HPLC): 11.6 min; ¹H NMR (500 MHz, D₂O) δ 0.92 (d, 6, J=5.5), 0.98-1.06 (m, 6), 1.40-1.60 (m, 4), 1.65-1.75 (m, 3), 1.75-1.85 (m, 1), 1.90-2.00 (m, 2), 2.02 (s, 3), 2.00-2.10 (m, 1), 2.28-2.35 (m, 1, J=7.5), 2.40-2.50 (m, 2), 2.98 (t, 2, J=7.5), 3.80-3.85 (m, 2), 4.25 (dd, 1), 4.30 (d, 1, J=18.6), 4.38 (dd, 1), 4.51 (d, 1, J=5.8), 4.73-4.80 (m, 1), 4.90 (t, 1, J=7.2), 5.69 (dd, 1, J=4.2, 11.6); MS (ES) m/z 656.5 (M+H⁺); HRMS (FAB) m/z 656.3636 (M+H⁺ C₂₉H₅₀N₇O₁₀ requires 656.3619).

### EXAMPLE 5

This example illustrates the generation of a peptide analog identical to that of Example 4 except that the [Leu] unit is replaced by a [^{D}Leu] unit, the superscript D denoting the (R)-stereoisomer. The synthesis begins with the D,L-diastereomer 10D of Section 4.A of Example 4.

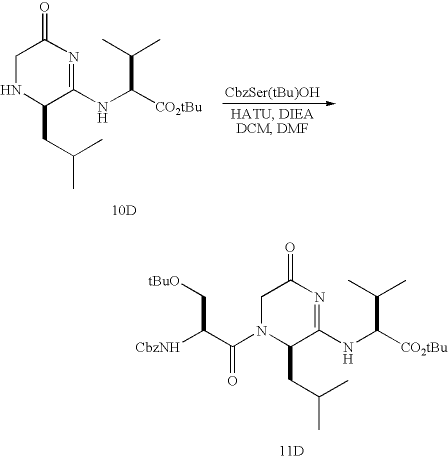

Following the procedure described in Section 4.B above, the product was obtained at 64% yield and 1/20 ratio of S/R stereoisomers. R_{f}=0.43 (UV, EtOAc/hexanes 1:1); retention time (analytical HPLC): 27.6 min; ¹H NMR (500 MHz, (CD₃)₂SO) δ 0.84-0.97 (m, 12), 1.09 (s, 9), 1.25-1.27 (m, 1), 1.37 (s, 9), 1.55-1.65 (m, 2), 2.12 (m, 1, J=6.8), 3.40-3.50 (m, 2), 3.90 (d, 1, J=17.6), 4.40 (dd, 1, J=7.2), 4.49 (d, 1, J=17.6), 4.53-4.58 (m, 1), 4.97 (d, 1, J=11.3), 5.02 (d, 1, J=12.4), 5.49 (dd, 1, J=5.2, 11.8), 7.28-7.37 (m, 5), 7.47 (d, 1, J=7.9), 9.09 (d, 1, J=7.9); MS (ES) m/z 603.5 (M+H⁺); HRMS (FAB) m/z 603.3763 (M+H⁺ C₃₂H₅₁N₄O₇ requires 603.3758).

### 5.B. Cbz-Glu(O^{γ}tBu)-Ser(O^{β}tBu)-[^{D}Leu]-Val t-Butyl Ester (12D) From Cbz-Ser(O^{β}tBu)-[^{D}Leu]-Val t-Butyl Ester (11D)

Following the procedure described in Section 4.C above, the product was obtained with 75% yield over two steps, and a 1/20 ratio of S/R stereoisomers. R_{f}=0.18 (UV, EtOAc/hexanes 1:1); retention time (analytical HPLC): 29.2 min; ¹H NMR (500 MHz, (CD₃)₂SO, major rotamer 7:2) δ 0.83-0.94 (m, 12), 1.10 (s, 9), 1.21-1.26 (m, 1), 1.37 (s, 9), 1.39 (s, 9), 1.56-1.88 (m, 4), 2.13 (m, 1, J=6.5), 2.20 (dd, 2, J=7.0), 3.47 (d, 2, J=3.5), 3.90 (d, 1, J=17.8), 3.98-4.06 (m, 1), 4.42-4.47 (m, 2), 4.76-4.80 (m, 1), 5.00 (d, 1, J=12.3), 5.03 (d, 1, J=12.3), 5.51 (dd, 1, J=4.0, 11.5), 7.30-7.38 (m, 5), 7.47 (d, 1, J=8.3), 8.03 (d, 1, J=7.4), 9.09 (d, 1, J=8.2); (minor rotamer, representative peaks) δ 3.61 (d, 1, J=18.4), 4.13-4.18 (m, 1), 4.51 (d, 1, J=18.3), 4.66 (dd, 1, J=4.5), 5.10 (dd, 1, J=4.2, 10.9), 7.96 (d, 1, J=8.7), 9.20 (d, 1, J=8.7); MS (ES) m/z 788.5 (M+H⁺); HRMS (FAB) m/z 788.4824 (M+H⁺ C₄₁H₆₆N₅O₁₀ requires 788.4810).

### 5.C. Cbz-Lys(N^{ε}Boc)-Glu(O^{γ}tBu)-Ser(O^{β}tBu)-[^{D}Leu]-Val t-Butyl Ester (13D) from Cbz-Glu(O^{γ}tBu)-Ser(O^{β}tBu)-[^{D}Leu]-Val t-Butyl Ester (12D)

Following the procedure described in Section 4.D above, the product was obtained with 75% yield over two steps, and a 1/20 ratio of S/R stereoisomers. R_{f}=0.10 (UV, EtOAc/hexanes 1:1); retention time (analytical HPLC): 30.3 min; ¹H NMR (500 MHz, (CD₃)₂SO, major rotamer 2:1) δ 0.84-0.94 (m, 12), 1.09 (s, 9), 1.20-1.30 (m, 5), 1.30-1.43 (m, 27), 1.45-1.50 (m, 1), 1.50-1.70 (m, 4), 1.80-1.90 (m, 1), 2.10-2.25 (m, 3), 2.80-2.90 (m, 2), 3.45 (d, 2, J=5.9), 3.90 (d, 1, J=17.8), 3.90-4.00 (m, 1), 4.30 (dd, 1, J=8.5, 13.6), 4.40-4.50 (m, 2), 4.77 (dd, 1, J=7.6, 14.5), 5.02 (s, 2), 5.50 (dd, 1, J=4.0, 11.2), 6.73 (t, 1, J=6.0), 7.25-7.38 (m, 6), 7.95 (d, 1, J=8.1), 8.06 (d, 1, J=7.3), 9.09 (d, 1, J=8.2); (minor rotamer, representative peaks) δ 1.70-1.80 (m, 1), 3.25-3.35 (m, 2), 3.60 (d, 1, J=18.4), 4.35-4.40 (m, 1), 4.50 (d, 1, J=18.4), 4.65 (dd, 1, J=4.9, 8.7), 4.95-5.05 (m, 1), 5.10 (dd, 1, J=11.3), 7.93 (d, 1, J=8.8), 8.01 (d, 1, J=10.0), 9.16 (d, 1, J=8.7); MS (ES) m/z 1016.8 (M+H⁺); HRMS (FAB) m/z 1016.6284 (M+H⁺ C₅₂H₈₆N₇O₁₃ requires 1016.6284).

### 5.D. Ac-Lys(N^{ε}Boc)-Glu(O^{γ}tBu)-Ser(O^{β}tBu)-[^{D}Leu]-Val t-Butyl Ester (14D) from Cbz-Lys(N^{ε}Boc)-Glu(O^{γ}tBu)-Ser(O^{β}tBu)-[^{D}Leu]-Val t-Butyl Ester (13D)

Following the procedure described in Section 4.E above, the product was obtained in quantitative yield over two steps, and a 1/20 ration of S/R stereoisomers. R_{f}=0.44 (UV, EtOH/EtOAc/TEA 10:90:1); retention time (analytical HPLC): 27.1 min; ¹H NMR (500 MHz, (CD₃)₂SO, major rotamer 3:1) δ 0.84-0.94 (m, 12), 1.09 (s, 9), 1.15-1.27 (m, 5), 1.30-1.42 (m, 2), 1.36 (s, 9), 1.37 (s, 9), 1.39 (s, 9), 1.52-1.70 (m, 3), 1.79-1.88 (m, 1), 1.83 (s, 3), 2,07-2.19 (m, 3), 2.83-2.91 (m, 2), 3.45 (d, 2, J=5.9), 3.89 (d, 1, J=17.8), 4.16-4.22 (m, 1), 4.26 (dd, 1, J=8.2, 12.8), 4.43 (dd, 1, J=5.8, 8.2), 4.44 (d, 1, J=17.6), 4.76 (dd, 1, J=6.0, 13.4), 5.49 (dd, 1, J=4.0, 11.3), 6.76 (dd, 1, J=5.6, 11.3), 7.96-8.01 (m, 3), 9.10 (d, 1, J=8.2); (minor rotamer, representative peaks) δ 1.95 (s, 3), 3.61 (d, 1, J=18.3), 4.34 (dd, 1), 4.49 (d, 1, J=18.5), 4.65 (dd, 1, J=4.8, 8.8), 5.03 (dd, 1, J=6.9, 14.4), 5.10 (dd, 1, J=11.5), 7.90 (d, 1, J=7.9), 8.05 (d, 1, J=8.3), 9.19 (d, 1, J=8.7); ¹³C NMR (125 MHz, (CD₃)₂SO) δ 18.4, 19.2, 20.9, 22.0, 22.9, 23.2, 23.5, 23.7, 24.3, 27.2, 27.3, 28.0, 28.1, 28.7, 29.7, 30.5, 31.7, 32.0, 45.8, 47.0, 51.7, 52.9, 59.0, 62.1, 67.0, 73.3, 77.7, 80.1, 81.5, 81.9, 155.9, 169.4, 169.7, 170.0, 171.1, 172.1, 172.4, 173.1, 175.8; MS (ES) m/z 924.7 (M+H⁺); HRMS (FAB) m/z 924.6011 (M+H⁺ C₄₆H₈₂N₇O₁₂ requires 924.6021).

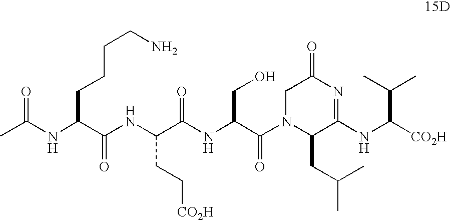

Following the procedure of Section 4.F above, the product was obtained in 58% yield, with a single (R) stereoisomer. Retention time (analytical HPLC): 13.8 min; ¹H NMR (500 MHz, (CD₃)₂SO, rotamers) δ 0.84-0.89 (m, 12), 1.20-1.35 (m, 4), 1.40-1.70 (m, 5), 1.70-1.90 (m, 2), 1.82 (s, 1.8), 1.83 (s, 1.2), 2.00-2.20 (m, 3), 2.65-2.75 (m, 2), 3.85 (dd, 1, J=3.4, 17.8), 3.87 (d, 1, J=17.5), 4.13-4.25 (m, 3), 4.45 (d, 1, J=17.8), 4.60 (m, 1), 4.60-4.70 (m, 1), 5.48 (dd, 1, J=4.4, 10.7), 8.00-8.05 (m, 1), 8.11 (s, 1), 8.28 (d, 0.3, J=8.0), 8.35 (d, 0.3, J=8.0), 8.52 (s, 0.6); MS (ES) m/z 656.5 (M+H⁺); HRMS (FAB) m/z 656.3636 (M+H⁺ C₂₉H₅₀N₇O₁₀ requires 656.3619).

### EXAMPLE 6

This example illustrates the liquid-phase synthesis of a peptide analog with protected side chains in which α-substituted dihydropyrazinones alternate with amino acid residues. As in the preceding examples, the dihydropyrazinone unit is denoted by a three-letter code in brackets, the three-letter code being that of the amino acid that bears the same side chain. The side chains on the dihydropyrazinone units in this peptide analog are those of leucine and the t-butyl ester of glutamic acid, and are thus abbreviated as [Leu] and [Glu(OtBu)]. The synthesis begins with reactions analogous to those of Example 1.

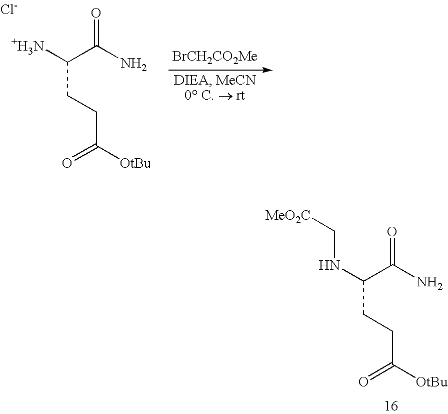

To a solution of 1.0 g (4.2 mmol) of L-glutamic acid γ-t-butyl ester amide hydrochloride and 1.6 mL (9.2 mmol) of DIEA in 17 mL of dry MeCN at 0° C. was slowly added a solution of methyl bromoacetate (0.44 mL, 4.6 mmol) in dry MeCN (3 mL). The reaction mixture was stirred under argon and allowed to warm up to room temperature over a 15-hour period before it was diluted with 15 mL of saturated NH₄Cl. The aqueous layer was then made basic with 25 mL of saturated NaHCO₃ and the product was extracted with EtOAc (2×50 mL). The organic layer was dried over Na₂SO₄, filtered, and concentrated to afford 16 as a white solid (0.92 g, 80% yield). R_{f}=0.41 (KMnO₄/basic, EtOAc/MeOH/TEA 90:10:1); ¹H NMR (500 MHz, CDCl₃) δ 1.18 (s, 9), 1.64 (m, 1, J=7.5), 1.72-1.76 (m, 1), 2.16 (t, 2, J=7.5), 3.08 (d, 1, J=17.5), 3.22 (d, 1, J=17.5), 3.46 (s, 3), 6.77 (d, 1, J=3.5), 7.07 (d, 1, J=3.5); MS (ES) m/z 275.3 (M+H⁺), 219.1 (M+H⁺-tBu); HRMS (FAB) m/z 275.1608 (M+H⁺ C₁₂H₂₃N₂O₅ requires 275.1607).

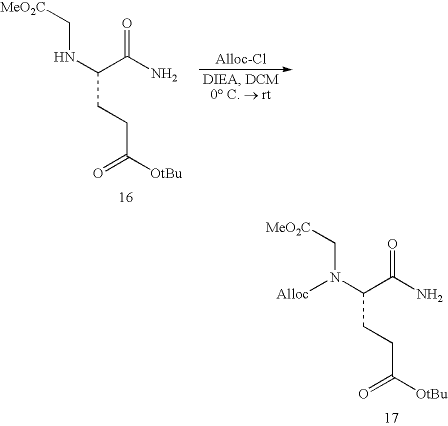

To a solution of 0.70 g (2.6 mmol) of 16 and 1.8 mL (10.2 mmol) of DIEA in 20 mL of dry DCM at 0° C. was slowly added 0.81 mL (7.7 mmol) of allyl chloroformate. The reaction solution was stirred under argon and allowed to warm up to room temperature over a 15-hour period before it was diluted with 30 mL of saturated NH₄Cl. An additional 30 mL of DCM was added to the organic layer, which was further washed with saturated NH₄Cl (2×15 mL) and saturated NaHCO₃ (2×15 mL), then dried over Na₂SO₄, filtered, and concentrated. The crude product was purified by flash chromatography (EtOAc/hexanes 1:2→1:1→2:1→3:1→4:1) to afford 17 as a yellow oil (0.73 g, 79% yield). R_{f}=0.35 (KMnO₄/basic, EtOAc/hex 10:3); ¹H NMR (500 MHz, CDCl₃, rotamers) δ 1.23 (s, 9), 1.69-1.79 (m, 1), 2.00-2.20 (m, 3), 3.53 (s, 1.6) 3.57 (s, 1.4), 3.72 (d, 0.5, J=17.7), 3.83 (d, 0.5, J=18.0), 3.92 (d, 0.5, J=17.2), 4.00-4.05 (m, 0.5), 4.08 (d, 0.5, J=17.7), 4.34 (dd, 0.5, J=5.8, 9.0), 4.35-4.42 (m, 2, J=5.4), 4.97-5.13 (m, 2), 5.66 (m, 1, J=5.3), 6.40 (s, 0.5), 6.43 (s, 0.5), 7.12 (s, 0.5), 7.55 (s, 0.5); ³C NMR (125 MHz, CDCl₃, rotamers) δ 20.4, 23.8, 24.4, 27.5, 27.5, 31.3, 31.4, 46.3, 51.8, 52.0, 59.8, 66.1, 66.4, 79.8, 80.0, 116.9, 117.5, 131.5, 131.7, 155.0, 155.1, 170.5, 170.6, 171.7, 171.8, 172.3, 172.5; MS (ES) m/z 381.2 (M+Na⁺), 258.1 (M+Na⁺-tBu-CONH₂); HRMS (FAB) m/z 359.1819 (M+H⁺ C₁₆H₂₇N₂O₇ requires 359.1818).

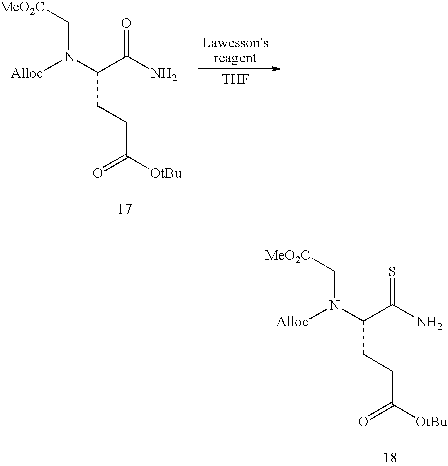

To 0.6 g (1.7 mmol) of 17 in 20 mL of dry THF was added 0.34 g (0.8 mmol) of Lawesson's reagent in two portions. The reaction solution was stirred under nitrogen, initially at 0° C. then with warming to room temperature. The reaction was stopped at 6 hours when no more starting material was observed by thin layer chromatography. After concentration of the reaction mixture, the crude product was purified by flash chromatography (EtOAc/hexanes 1:3→1:2→2:3) to afford 18 as a white solid (0.50 g, 80% yield). R_{f}=0.49 (UV, KMnO₄, EtOAc/hexanes 1:1); ¹H NMR (500 MHz, CDCl₃, rotamers) δ 1.45 (s, 9), 2.02-2.09 (m, 1), 2.29-2.35 (m, 2), 2.60-2.70 (m, 0.6), 2.90-3.00 (m, 0.4), 3.75 (s, 1.8), 3.81 (s, 1.2), 3.98 (d, 1, J=18.0), 4.14 (d, 1, J=18.0), 4.60 (d; 1, J=5.5), 4.63 (d, 1, J=5.5), 4.60-4.65 (m, 0.4), 4.83 (dd, 0.6, J=6.5), 5.21-5.35 (m, 2), 5.85-5.90 (m, 1), 7.79 (s, 0.5), 7.84 (s, 0.4), 8.62 (s, 0.5), 9.38 (s, 0.4); ¹³C NMR (125 MHz, CDCl₃, rotamers) δ 26.7, 27.8, 28.0, 32.0, 32.3, 46.2, 52.5, 52.9, 63.7, 65.7, 66.9, 67.2, 80.7, 80.9, 117.8, 118.5, 131.6, 131.9, 155.6, 156.2, 171.0, 171.9, 172.4, 205.8, 206.3; MS (ES) m/z 397.2 (M+Na⁺), 319.2 (M+H⁺-tBu); HRMS (FAB) m/z 375.1583 (M+H⁺ C₁₆H₂₇N₂O₆S requires 375.1590).

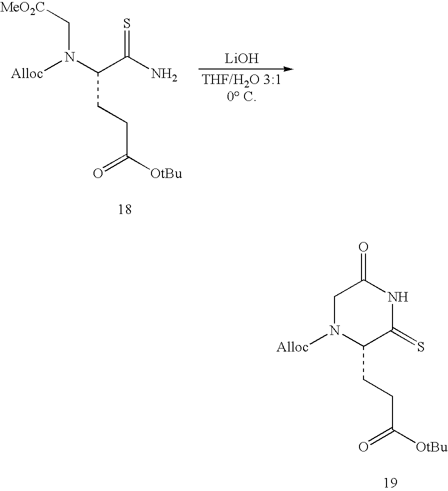

To 0.29 g (0.78 mmol) of 18 in 20 mL of THF/H₂O 3:1 at 0° C. was added 1 M LiOH (0.78 mL, 0.78 mmol), which turned the solution bright yellow. The reaction solution was stirred under nitrogen and quenched after 15 minutes by addition of solid KHSO₄. After concentration of the reaction mixture, the aqueous solution was extracted with EtOAc (2×30 mL). The organic layer was further washed with 1 M KHSO₄ (1×15 mL), saturated NaHCO₃ (1×15 mL), and brine (1×10 mL), dried over Na₂SO₄, filtered, and concentrated. The crude product was purified by flash chromatography (EtOAc/hexanes 1:3) to afford 19 as a yellow oil (0.22 g, 83% yield, >99% e.e.). R_{f}=0.58 (UV, EtOAc/hexanes 1:1); retention time (analytical HPLC): 24.8 min; retention time (chiral HPLC): 8.3 min; ¹H NMR (500 MHz, CDCl₃) δ 1.41 (s, 9), 2.10-2.17 (m, 1), 2.23-2.36 (m, 3), 3.80-4.10 (m, 1), 4.61 (s, 2), 4.80-5.00 (m, 1), 5.24 (dd, 1, J=1.0, 10.5), 5.30 (dd, 1, J=1.0, 17.0), 5.89 (m, 1, J=5.5, 10.5, 16.0), 10.24 (s, 1); ¹³C NMR (125 MHz, CDCl₃) δ 27.6, 27.9, 31.7, 42.2, 42.7, 52.5, 61.8, 62.4, 67.2, 81.0, 81.1, 118.8, 131.7, 153.8, 156.6, 164.9, 168.6, 171.3, 171.8, 203.6, 205.1; MS (ES) m/z 365.2 (M+Na⁺), 309.1 (M+Na⁺-tBu).

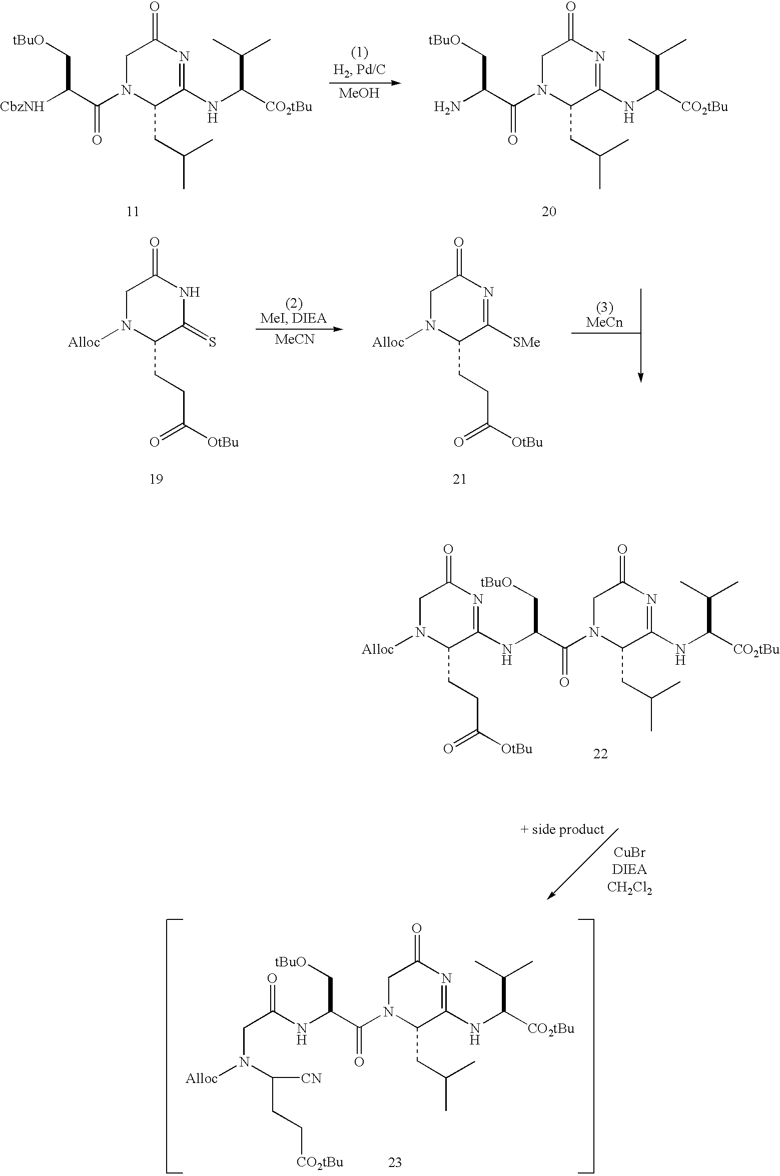

Reaction (1): To 0.20 g (0.34 mmol) of 11 in 30 mL of MeOH under an atmosphere of argon was added 36 mg (0.03 mmol) of 10% wt Pd/C. The argon gas was removed with vacuum and replaced with an atmosphere of hydrogen. Hydrogen gas was bubbled through the stirred reaction solution for 30 min, after which the solution was filtered through Celite and concentrated. The crude product, 20, was used without further purification.

Reaction (2): To 0.17 g (0.5 mmol) of 19 in 5 mL of MeCN under an atmosphere of argon at 0° C. was added 0.19 mL (3.0 mmol) of methyl iodide followed by 88 μL (0.5 mmol) of DIEA. The reaction solution was then sealed and stirred for 1 hour. When no more starting material was observed, the reaction was stopped and the solvent and excess methyl iodide were removed by rotary evaporation to give the acyl thioimidate 21.

Reaction (3) The crude amine 20 was dissolved in 5 mL of MeCN and added to excess acyl thioimidate 21. The reaction solution was stirred under nitrogen at room temperature for 20 hours. The reaction mixture was then concentrated and the crude product was purified by flash chromatography (EtOAc/hexanes 1:1→2:1→3:1→5:1 EtOAc) to afford a mixture of the desired product 22 and a side product. The side product was converted to the nitrile 23 upon addition of CuBr and DIEA in DCM, and then separated easily from the desired product, 22, by flash chromatography (EtOAc/hexanes 1:1→3:1→6:1→EtOAc0; 22 was obtained as a clear oil (94.4 mg, 36% yield over 3 steps). R_{f}=0.27 (UV, EtOAc); ¹H NMR (500 MHz, (CD₃)₂SO) δ 0.82 (d, 3, J=6.5), 0.86 (d, 3, J=6.5), 0.90 (d, 3, J=7.0), 0.94 (d, 3, J=7.0), 0.99 (s, 9), 1.25-1.40 (m, 1), 1.37 (s, 9), 1.41 (s, 9), 1.55-1.60 (m, 2), 1.75-1.80 (m, 2), 2.10-2.20 (m, 1), 2.20-2.30 (m, 2), 3.40-3.50 (m, 1), 3.55-3.60 (m, 1), 3.75 (d, 0.5), 3.85 (d, 0.5), 3.97 (d, 1, J=17.5), 4.13 (d, 1, J=18.5), 4.38-4.39 (m, 1), 4.42 (d, 1, J=17.0), 4.55 (m, 2), 4.80-4.90 (m, 1), 4.90-5.00 (m, 1), 5.20-5.40 (m, 2), 5.44 (dd, 1, J=3.5, 11.5), 5.85-6.00 (m, 1), 8.90 (d, 1, J=8.0), 9.02 (d, 0.5), 9.05 (d, 0.5); MS (ES) m/z 777.6 (M+H⁺); ¹³C NMR (125 MHz, (CD₃)₂SO, rotamers) δ 18.1, 18.9, 21.4, 23.4, 24.2, 26.8, 27.6, 27.7, 29.7, 31.4, 45.0, 46.5, 59.2, 50.3, 58.9, 61.3, 65.8, 73.2, 79.9, 81.1, 117.2, 117.4, 132.9, 153.7, 168.9, 169.3, 170.1, 170.3, 171.2, 172.6, 175.1, 175.6; HRMS (FAB) m/z 777.4743 (C₃₉H₆₅N₆O₁₀ requires 777.4762).

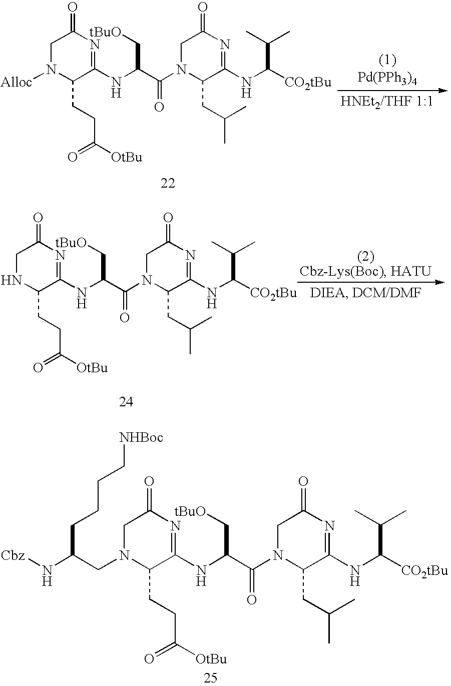

Reaction (1): To 94 mg (0.12 mmol) of 22 in 2 mL of dry THF was added 2 mL of diethylamine, followed by Pd(PPh₃)₄ (28 mg, 0.02 mmol). The reaction solution was stirred under nitrogen at room temperature for 1 hour. After concentration of the reaction mixture, the crude amine 24 was purified by flash chromatography (EtOAc/hexanes/TEA 75:25:1→EtOAc/TEA 100:1→EtOH/EtOAc/TEA 5:95:1→10:90:1) to afford 24 as a yellow oil (78 mg, 92%). R_{f}=0.09 (UV, EtOH/EtOAc 1:9); MS (ES) m/z 693.6 (M+H⁺).

Reaction (2): To 24 in 5 mL of dry DCM and 1 mL of dry DMF was added 92 mg (0.24 mmol) of Cbz-Lys(Boc) followed by 43 μL (0.24 mmol) of DIEA and 92 mg (0.24 mmol) of HATU. The reaction solution was stirred under nitrogen at room temperature for 18 hours, then the mixture was concentrated and the crude product was redissolved in EtOAc. The organic layer was washed with NaH₂PO₄ (1×15 mL), saturated NaHCO₃ (1×15 mL), and brine (1×10 mL), dried over Na₂SO₄, filtered, and concentrated. The crude product was purified by flash chromatography (EtOAc/hexanes 1:1→3:1→EtOAc a EtOH/EtOAc 5/95) to afford 25 as a white solid (87 mg, 74% yield). R_{f}=0.28 (UV, EtOAc); ¹H NMR (500 MHz, (CD₃)₂SO) δ 0.82 (d, 3, J=6.5), 0.87 (m, 3), 0.90 (d, 3, J=7.0), 0.94 (d, 3, J=7.0), 1.00 (s, 9), 1.20-1.50 (m, 3), 1.36 (s, 9), 1.37 (s, 9), 1.41 (s, 9), 1.50-1.65 (m, 6), 1.70-1.80 (m, 2), 2.10-2.20 (m, 2), 2.25-2.35 (m, 1), 2.80-2.95 (m, 2), 3.50-3.60 (m, 2), 3.90-4.10 (m, 2), 4.20-4.30 (m, 2), 4.35-4.50 (m, 2), 4.90-5.00 (m, 1), 5.02 (s, 2), 5.13 (dd, 1), 5.45 (dd, 1), 6.76 (t, 1), 7.30-7.35 (m, 5), 8.92 (d, 1, J=8.0), 9.09 (d, 1, J=6.5); MS (ES) m/z 1055 (M+H⁺).

The foregoing is offered primarily for purposes of illustration. Further modifications and variations that still embody the underlying concepts of this invention and fall within its scope will be apparent to those skilled in the art.

1. A peptide analog comprising a peptide in which at least one amino acid, but less than all amino acids, is replaced by an azacyclohexenone group having the formula 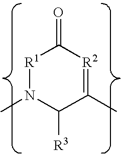
in which:
R¹ is CH₂ or NH,
R² is CH or N, and
R³ is H or an amino acid side chain,
such that in at least one such azacyclohexenone group:
when R¹ is CH₂ and R² is CH, R³ is an amino acid side chain, and
when either R¹ is NH, or R² is N, or R¹ is NH and R² is N, R³ is H or an amino acid side chain,
and when said peptide analog contains two or more azacyclohexenone groups of said formula, R¹, R², and R³ of any one azacyclohexenone group in said peptide analog are either the same as or different from R¹, R², and R³ of any other azacyclohexenone group in said peptide analog.

2. A peptide analog comprising a peptide in which at least one amino acid, but less than all amino acids, is replaced by an azacyclohexenone group having the formula 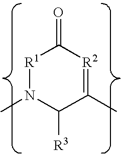
in which:
R¹ is CH₂ or NH,
R² is CH or N, and
when R¹ is CH₂ and R² is CH, R³ is an amino acid side chain, and
when either R¹ is NH, or R² is N, or R³ is NH and R² is N, R³ is H or an amino acid side chain,
and when said peptide analog contains two or more azacyclohexenone groups of said formula, R¹, R², and R³ of any one azacyclohexenone group in said peptide analog are either the same as or different from R¹, R², and R³ of any other azacyclohexenone group in said peptide analog.

3. The peptide analog of claims 1 or 2 in which R¹ is CH₂ and R² is N.

4. The peptide analog of claims 1 or 2 in which R¹ is NH and R² is CH.

5. The peptide analog of claims 1 or 2 in which R¹ is NH and R² is N.

6. The peptide analog of claims 1 or 2 in which R¹ is CH₂ and R² is CH.

7. The peptide analog of claims 1 or 2 in which said azacyclohexenone group is an L-stereoisomer relative to R³ when R³ is an amino acid side chain.

8. The peptide analog of claims 1 or 2 in which said amino acid side chain is a side chain of a natural amino acid.

9. The peptide analog of claims 1 or 2 in which said amino acid side chain is a side chain of an unnatural amino acid.

10. The peptide analog of claims 1 or 2 in which said amino acid side chain is a member selected from the group consisting of C₁-C₆ alkyl, C₁-C₆ alkyl interrupted by —O—, C₁-C₆ alkyl interrupted by —S—, hydroxy-(C₁-C₆ alkyl), carboxy-(C₁-C₆ alkyl), amino-(C₁-C₆ alkyl), guanidino-(C₁-C₆ alkyl), carbamoyl-(C₁-C₆ alkyl), mercapto-(C₁-C₆ alkyl), indolyl-(C₁-C₃ alkyl), phenyl-(C₁-C₃ alkyl), hydroxyphenyl-(C₁-C₆ alkyl), halophenyl-(C₁-C₆ alkyl), imidazolyl-(C₁-C₆ alkyl), phenyl, and sulfoximino-(C₁-C₆ alkyl).

11. The peptide analog of claims 1 or 2 in which said amino acid side chain is a member selected from the group consisting of C₁-C₄ alkyl, hydroxy-(C₁-C₂ alkyl), carboxy-(C₁-C₂ alkyl), amino-(C₃-C₅ alkyl), guanidino-(C₂-C₄ alkyl), carbamoyl-(C₁-C₂ alkyl), mercapto-(C₁-C₂ alkyl), methylthio-(C₁-C₃ alkyl), indolylmethyl, phenyl-(C₁-C₂ alkyl), and hydroxyphenyl-(C₁-C₂ alkyl).

12. The peptide analog of claims 1 or 2 in which R¹ is CH₂, R² is N, and said amino acid side chain is a member selected from the group consisting of C₁-C₄ alkyl, hydroxy-(C₁-C₂ alkyl), carboxy-(C₁-C₂ alkyl), amino-(C₃-C₅ alkyl), guanidino-(C₂-C₄ alkyl), carbamoyl-(C₁-C₂ alkyl), mercapto-(C₁-C₂ alkyl), methylthio-(C₁-C₃ alkyl), indolylmethyl, phenyl-(C₁-C₂ alkyl), and hydroxyphenyl-(C₁-C₂ alkyl).

13. The peptide analog of claims 1 or 2 in which the amino acids of said peptide analog are from 2 to 200 in number and said azacyclohexenone groups are from 1 to 100 in number.

14. The peptide analog of claims 1 or 2 in which the amino acids of said peptide analog are from 2 to 200 in number, said azacyclohexenone groups are from 1 to 100 in number, and the number ratio of said azacyclohexenone groups to amino acids is from 1:10 to 10:1.

15. The peptide analog of claims 1 or 2 in which the amino acids of said peptide analog are from 2 to 100 in number and said azacyclohexenone groups are from 1 to 50 in number.

16. The peptide analog of claims 1 or 2 in which the amino acids of said peptide analog are from 2 to 100 in number, said azacyclohexenone groups are from 1 to 50 in number, and the number ratio of said azacyclohexenone groups to amino acids is from 1:10 to 10:1.

17. The peptide analog of claims 1 or 2 in which all remaining amino acids in said peptide analog are a combination of natural and unnatural amino acids.

18. The peptide analog of claims 1 or 2 in which all remaining amino acids in said peptide analog are natural amino acids.

19. The peptide analog of claims 1 or 2 in which R¹ is CH₂, R² is N, and R³ is a member selected from the group consisting of C₁-C₄ alkyl, hydroxy-(C₁-C₂ alkyl), carboxy-(C₁-C₂ alkyl), amino-(C₃-C₅ alkyl), guanidino-(C₂-C₄ alkyl), carbamoyl-(C₁-C₂ alkyl), mercapto-(C₁-C₂ alkyl), methylthio-(C₁-C₃ alkyl), indolylmethyl, phenyl-(C₁-C₂ alkyl), and hydroxyphenyl-(C₁-C₂ alkyl), and all remaining amino acids in said peptide analog are natural amino acids.

20. A peptide analog having the formula 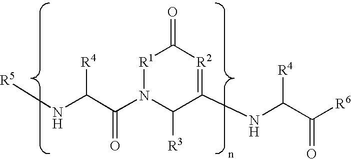
in which:
R¹ is CH₂ or NH,
R² is CH or N,
R³ is H or an amino acid side chain,
such that in at least one such azacyclohexenone group:
when R¹ is CH₂ and R² is CH, R³ is an amino acid side chain, and
when either R¹ is NH, or R² is N, or R¹ is NH and R² is N, R³ is H or an amino acid side chain,
and when said peptide analog contains two or more azacyclohexenone groups of said formula, R¹, R², and R³ of any one azacyclohexenone group in said peptide analog are either the same as or different from R¹, R², and R³ of any other azacyclohexenone group in said peptide analog,
the R⁴'s are the same or different and each R⁴ is either H or an amino acid side chain,
R⁵ is a member selected from the group consisting of peptide chain terminating groups and 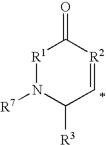
in which R⁷ is a member selected from the group consisting of H, alkyl, acyl, carbamoyl, and alkoxycarbamoyl, and * denotes the site of attachment,
R⁶ is a member selected from the group consisting of peptide chain terminating groups and 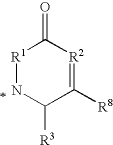
in which R⁸ is a member selected from the group consisting of hydroxyl, alkoxy, alkylamino, dialkylamino, and arylamino, and * denotes the site of attachment, and
n is at least 2.

21. A peptide analog having the formula 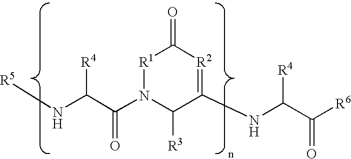
in which:
R¹ is CH₂ or NH,
R² is CH or N,
when R¹ is CH₂ and R² is CH, R³ is an amino acid side chain, and
when either R¹ is NH, or R² is N, or R³ is NH and R² is N, R³ is H or an amino acid side chain,
and when said peptide analog contains two or more azacyclohexenone groups of said formula, R¹, R², and R³ of any one azacyclohexenone group in said peptide analog are either the same as or different from R¹, R², and R³ of any other azacyclohexenone group in said peptide analog,
the R⁴'s are the same or different and each R⁴ is either H or an amino acid side chain,
R⁵ is a member selected from the group consisting of peptide chain terminating groups and 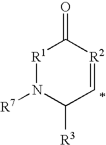
in which R⁷ is a member selected from the group consisting of H, alkyl, acyl, carbamoyl, and alkoxycarbamoyl, and * denotes the site of attachment,
R⁶ is a member selected from the group consisting of peptide chain terminating groups and 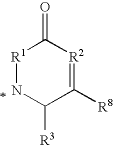
in which R⁸ is a member selected from the group consisting of hydroxyl, alkoxy, alkylamino, dialkylamino, and arylamino, and * denotes the site of attachment, and
n is at least 2.

22. The peptide analog of claim 21 in which R¹ is CH₂ and R² is N.

23. The peptide analog of claim 21 in which R¹ is NH and R² is CH.

24. The peptide analog of claim 21 in which R¹ is NH and R² is N.

25. The peptide analog of claim 21 in which R¹ is CH₂ and R² is CH.

26. The peptide analog of claim 21 in which said peptide analog is an L-stereoisomer relative to R³ when R³ is an amino acid side chain

27. The peptide analog of claim 21 in which all R³'s are side chains of natural amino acids.

28. The peptide analog of claim 21 in which at least one R³ is a side chain of a natural amino acid.

29. The peptide analog of claim 21 in which each R⁴ is either H or a side chain of a natural amino acid.

30. The peptide analog of claim 21 in which at least one R⁴ is either H or a side chain of a natural amino acid.

31. The peptide analog of claim 21 in which all R³'s and all R⁴'s are members selected from the group consisting of H, C₁-C₆ alkyl, C₁-C₆ alkyl interrupted by —O—, C₁-C₆ alkyl interrupted by —S—, hydroxy-(C₁-C₆ alkyl), carboxy-(C₁-C₆ alkyl), amino-(C₁-C₆ alkyl), guanidino-(C₁-C₆ alkyl), carbamoyl-(C₁-C₆ alkyl), mercapto-(C₁-C₆ alkyl), indolyl-(C₁-C₃ alkyl), phenyl-(C₁-C₃ alkyl), hydroxyphenyl-(C₁-C₆ alkyl), halophenyl-(C₁-C₆ alkyl), imidazolyl-(C₁-C₆ alkyl), phenyl, and sulfoximino-(C₁-C₆ alkyl).

32. The peptide analog of claim 21 in which all R³'s and all R⁴'s are members selected from the group consisting of H, C₁-C₄ alkyl, hydroxy —(C₁-C₂ alkyl), carboxy-(C₁-C₂ alkyl), amino-(C₃-C₅ alkyl), guanidino —(C₂-C₄ alkyl), carbamoyl-(C₁-C₂ alkyl), mercapto-(C₁-C₂ alkyl), methylthio-(C₁-C₃ alkyl), indolylmethyl, phenyl-(C₁-C₂ alkyl), and hydroxyphenyl-(C₁-C₂ alkyl).

33. The peptide analog of claim 21 in which R¹ is CH₂, R² is N, and all R³'s and all R⁴'s are members selected from the group consisting of H, C₁-C₄ alkyl, hydroxy —(C₁-C₂ alkyl), carboxy-(C₁-C₂ alkyl), amino-(C₃-C₅ alkyl), guanidino —(C₂-C₄ alkyl), carbamoyl-(C₁-C₂ alkyl), mercapto-(C₁-C₂ alkyl), methylthio-(C₁-C₃ alkyl), indolylmethyl, phenyl-(C₁-C₂ alkyl), and hydroxyphenyl-(C₁-C₂ alkyl).

34. The peptide analog of claim 21 in which the R⁴'s are a combination comprising side chains of natural and unnatural amino acids.

35. The peptide analog of claim 21 in which each R⁴ is either H or a side chain of a natural amino acid.

36. The peptide analog of claim 21 in which all remaining amino acids in said peptide analog are a combination comprising natural and unnatural amino acids.

37. The peptide analog of claim 21 in which all remaining amino acids in said peptide analog are natural amino acids.

38. The peptide analog of claim 21 in which R⁵ is a member selected from the group consisting of H, alkyl, acyl, carbamoyl, and alkoxycarbonyl.

39. The peptide analog of claim 21 in which R⁵ is acetyl.

40. The peptide analog of claim 21 in which R⁵ is

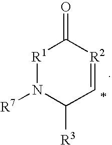

41. The peptide analog of claim 21 in which R⁶ is a member selected from the group consisting of hydroxyl, alkoxy, alkylamino, dialkylamino, and arylamino.

42. The peptide analog of claim 21 in which R⁶ is a member selected from the group consisting of hydroxyl and methylamino.

43. The peptide analog of claim 21 in which R⁶ is

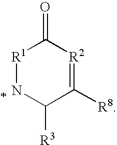

44. The peptide analog of claim 21 in which n is 2 to 100.

45. The peptide analog of claim 21 in which n is 2 to 50.

46. The peptide analog of claim 21 in which n is 2 to 5.

47. The peptide analog of claim 33 in which R⁵ is a member selected from the group consisting of H, alkyl, acyl, carbamoyl, and alkoxycarbonyl, and R⁶ is a member selected from the group consisting of hydroxyl, alkoxy, alkylamino, dialkylamino, and arylamino.

48. The peptide analog of claim 33 in which R⁵ is

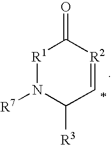

49. The peptide analog of claim 33 in which R⁶ is

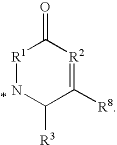

50. A peptide analog comprising a first segment consisting of a first sequence of amino acids joined by amide bonds and a second segment consisting of a second sequence of amino acids joined by amide bonds, in which at least one amino acid, but less than all amino acids, of said second segment is replaced by an azacyclohexenone group having the formula 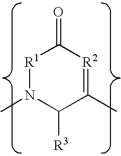
in which:
R¹ is CH₂ or NH,
R² is CH or N, and
R³ is H or an amino acid side chain,
such that in at least one such azacyclohexenone group:
when R¹ is CH₂ and R² is CH, R³ is an amino acid side chain, and
when either R¹ is NH, or R² is N, or R¹ is NH and R² is N, R³ is H or an amino acid side chain,
and when said peptide analog contains two or more azacyclohexenone groups of said formula, R¹, R², and R³ of any one azacyclohexenone group in said peptide analog are either the same as or different from R¹, R², and R³ of any other azacyclohexenone group in said peptide analog,
said first and second segments joined by a covalent linkage that permits said first and second segments to enter into a β-sheet-like interaction with each other or with a third sequence of amino acids joined by amide bonds.

51. A peptide analog comprising a first segment consisting of a first sequence of amino acids joined by amide bonds and a second segment consisting of a second sequence of amino acids joined by amide bonds, in which at least one amino acid, but less than all amino acids, of said second segment is replaced by an azacyclohexenone group having the formula 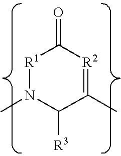
in which:
R¹ is CH₂ or NH,
R² is CH or N, and
when R¹ is CH₂ and R² is CH, R³ is an amino acid side chain, and
when either R¹ is NH, or R² is N, or R¹ is NH and R² is N, R³ is H or an amino acid side chain,
and when said peptide analog contains two or more azacyclohexenone groups of said formula, R¹, R², and R³ of any one azacyclohexenone group in said peptide analog are either the same as or different from R¹, R², and R³ of any other azacyclohexenone group in said peptide analog,
said first and second segments joined by a covalent linkage that permits said first and second segments to enter into a β-sheet-like interaction with each other or with a third sequence of amino acids joined by amide bonds.

52. The peptide analog of claim 50 in which R¹ is CH₂ and R² is N.

53. The peptide analog of claim 50 in which R¹ is NH and R² is CH.

54. The peptide analog of claim 50 in which R¹ is NH and R² is N.

55. The peptide analog of claim 50 in which R¹ is CH₂ and R² is CH.

56. The peptide analog of claim 50 in which said peptide analog is an L-stereoisomer relative to R³ when R³ is an amino acid side chain

57. The peptide analog of claim 50 in which all R³'s are side chains of natural amino acids.

58. The peptide analog of claim 50 in which at least one R³ is a side chain of a natural amino acid.

59. The peptide analog of claim 50 in which all R³'s are members selected from the group consisting of C₁-C₆ alkyl, C₁-C₆ alkyl interrupted by —O—, C₁-C₆ alkyl interrupted by —S—, hydroxy —(C₁-C₆ alkyl), carboxy-(C₁-C₆ alkyl), amino-(C₁-C₆ alkyl), guanidino —(C₁-C₆ alkyl), carbamoyl-(C₁-C₆ alkyl), mercapto-(C₁-C₆ alkyl), indolyl-(C₁-C₃ alkyl), phenyl-(C₁-C₃ alkyl), hydroxyphenyl-(C₁-C₆ alkyl), halophenyl-(C₁-C₆ alkyl), imidazolyl-(C₁-C₆ alkyl), phenyl, and sulfoximino-(C₁-C₆ alkyl).

60. The peptide analog of claim 50 in which all R³'s are members selected from the group consisting of C₁-C₄ alkyl, hydroxy —(C₁-C₂ alkyl), carboxy-(C₁-C₂ alkyl), amino-(C₃-C₅ alkyl), guanidino —(C₂-C₄ alkyl), carbamoyl-(C₁-C₂ alkyl), mercapto-(C₁-C₂ alkyl), methylthio-(C₁-C₃ alkyl), indolylmethyl, phenyl-(C₁-C₂ alkyl), and hydroxyphenyl-(C₁-C₂ alkyl).

61. The peptide analog of claim 50 in which R¹ is CH₂, R² is N, and all R³'s are members selected from the group consisting of C₁-C₄ alkyl, hydroxy —(C₁-C₂ alkyl), carboxy-(C₁-C₂ alkyl), amino-(C₃-C₅ alkyl), guanidino —(C₂-C₄ alkyl), carbamoyl-(C₁-C₂ alkyl), mercapto-(C₁-C₂ alkyl), methylthio-(C₁-C₃ alkyl), indolylmethyl, phenyl-(C₁-C₂ alkyl), and hydroxyphenyl-(C₁-C₂ alkyl).

62. The peptide analog of claim 50 in which the amino acids in said first segment are a combination of natural and unnatural amino acids.

63. The peptide analog of claim 50 in which the amino acids in said first segment are natural amino acids.

64. The peptide analog of claim 50 in which the remaining amino acids in said second segment are a combination of natural and unnatural amino acids.

65. The peptide analog of claim 50 in which the remaining amino acids in said second segment are natural amino acids.

66. The peptide analog of claim 50 in which said second segment consists of an amino acid sequence in which two or more non-adjacent amino acids are replaced by azacyclohexenone groups of said formula.

67. The peptide analog of claim 50 in which, in at least a portion of said second segment, every second amino acid is replaced by azacyclohexenone groups of said formula.

68. The peptide analog of claim 50 in which said first segment contains from 3 to 200 amino acids and in said second segment the total number of amino acids and azacyclohexenone groups is from 3 to 200.

69. The peptide analog of claim 50 in which said first segment contains from 3 to 20 amino acids and in said second segment the total number of amino acids and azacyclohexenone groups is from 3 to 20.

70. The peptide analog of claim 50 in which said covalent linkage is a member selected from the group consisting of D-Pro-Ala and Asn-Gly.

71. A compound having the formula 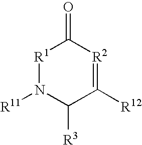
in which:
R¹ is CH₂ or NH,
R² is CH or N,
when R¹ is CH₂ and R² is CH, R³ is an amino acid side chain, when either R¹ is NH, or R² is N, or R¹ is NH and R² is N, R³ is H or an amino acid side chain,
R¹¹ is a nitrogen protecting group, and
R¹² is a member selected from the group consisting of OH, SH, and activated leaving groups.

72. The compound of claim 71 in which R¹ is CH₂ and R² is N.

73. The compound of claim 71 in which R¹ is NH and R² is CH.

74. The compound of claim 71 in which R¹ is NH and R² is N.

75. The compound of claim 71 in which R¹ is CH₂ and R² is CH.

76. The compound of claim 71 in which said compound is an L-stereoisomer relative to R³ when R³ is an amino acid side chain

77. The compound of claim 71 in which R³ is a side chain of a natural amino acid.

78. The compound of claim 71 in which R³ is a member selected from the group consisting of C₁-C₆ alkyl, C₁-C₆ alkyl interrupted by —O—, C₁-C₆ alkyl interrupted by —S—, hydroxy-(C₁-C₆ alkyl), carboxy-(C₁-C₆ alkyl), amino-(C₁-C₆ alkyl), guanidino-(C₁-C₆ alkyl), carbamoyl-(C₁-C₆ alkyl), mercapto-(C₁-C₆ alkyl), (C₁-C₃ alkyl)thio-(C₁-C₃ alkyl), indolyl-(C₁-C₃ alkyl), phenyl-(C₁-C₃ alkyl), hydroxyphenyl-(C₁-C₆ alkyl), halophenyl-(C₁-C₆ alkyl), imidazolyl-(C₁-C₆ alkyl), phenyl, and sulfoximino-(C₁-C₆ alkyl).

79. The compound of claim 71 in which R³ is a member selected from the group consisting of C₁-C₄ alkyl, hydroxy-(C₁-C₂ alkyl), carboxy-(C₁-C₂ alkyl), amino-(C₃-C₅ alkyl), guanidino-(C₂-C₄ alkyl), carbamoyl-(C₁-C₂ alkyl), mercapto-(C₁-C₂ alkyl), methylthio-(C₁-C₃ alkyl), indolylmethyl, phenyl-(C₁-C₂ alkyl), and hydroxyphenyl-(C₁-C₂ alkyl).

80. The compound of claim 71 in which R¹ is CH₂, R² is N, and R³ is a member selected from the group consisting of C₁-C₄ alkyl, hydroxy-(C₁-C₂ alkyl), carboxy-(C₁-C₂ alkyl), amino-(C₃-C₅ alkyl), guanidino-(C₂-C₄ alkyl), carbamoyl-(C₁-C₂ alkyl), mercapto-(C₁-C₂ alkyl), methylthio-(C₁-C₃ alkyl), indolylmethyl, phenyl-(C₁-C₂ alkyl), and hydroxyphenyl-(C₁-C₂ alkyl).

81. The compound of claim 80 in which R¹² is OH.

82. The compound of claim 80 in which R¹² is an activated leaving group.

83. A compound having a formula selected from the group consisting of 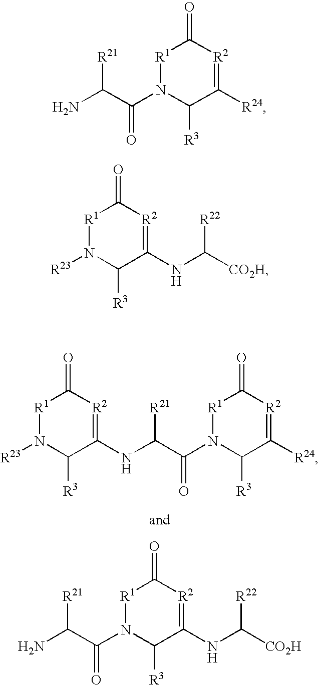
in which:
R¹ is CH₂ or NH,
R² is CH or N,
when R¹ is CH₂ and R² is CH, R³ is an amino acid side chain,
when either R¹ is NH, or R² is N, or R¹ is NH and R² is N, R³ is H or an amino acid side chain, and
when R¹, R², and R³ occur twice in said formula, each R¹ is either the same or different, each R² is either the same or different, and each R³ is either the same or different,
R²¹ is H or an amino acid side chain;
R²² is H or an amino acid side chain;
R²³ is a member selected from the group consisting of H and amine protecting groups; and
R²⁴ is a member selected from the group consisting of an activated leaving group, OR²⁵ where R²⁵ is H or an oxygen-protecting group, SR²⁶ where R²⁶ is H or an alkyl or aryl group, and N(R²⁷)₂, where the R²⁷'s are members independently selected from the group consisting of H, alkyl, and aryl;
and amine-protected analogs of those of said group that terminate in H₂N—, carboxy-protected analogs of those of said group that terminate in —CO₂H, carboxy-activated analogs of those of said group that terminate in —CO₂H, amine-protected and carboxy-protected analogs of 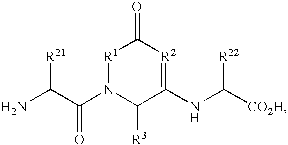
and amine-protected and carboxy-activated analogs of

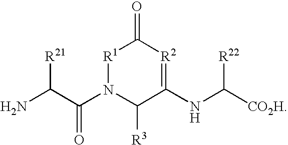

84. The compound of claim 83 in which R¹ is CH₂ and R² is N.

85. The compound of claim 83 in which R¹ is NH and R² is CH.

86. The compound of claim 83 in which R¹ is NH and R² is N.

87. The compound of claim 83 in which R¹ is CH₂ and R² is CH.

88. The compound of claim 83 in which said compound is an L-stereoisomer relative to R³ when R³ is an amino acid side chain.

89. The compound of claim 83 in which R³ is a side chain of a natural amino acid of a natural amino acid.

90. The compound of claim 83 in which R³ is a side chain of an unnatural amino acid of a natural amino acid.

91. The compound of claim 83 in which R³ is a side chain of a natural amino acid and R²¹ and R²² are independently H or side chains of natural amino acids.

92. The compound of claim 83 in which at least one of R³, R²′, and R²² is a side chain of a natural amino acid.

93. The compound of claim 83 in which R³, R²¹, and R²² are members selected from the group consisting of H, C₁-C₆ alkyl, C₁-C₆ alkyl interrupted by —O—, C₁-C₆ alkyl interrupted by —S—, hydroxy-(C₁-C₆ alkyl), carboxy-(C₁-C₆ alkyl), amino-(C₁-C₆ alkyl), guanidino-(C₁-C₆ alkyl), carbamoyl-(C₁-C₆ alkyl), mercapto-(C₁-C₆ alkyl), indolyl-(C₁-C₃ alkyl), phenyl-(C₁-C₃ alkyl), hydroxyphenyl-(C₁-C₆ alkyl), halophenyl-(C₁-C₆ alkyl), imidazolyl-(C₁-C₆ alkyl), phenyl, and sulfoximino-(C₁-C₆ alkyl).

94. The compound of claim 83 in which R³, R²¹, and R²² are members selected from the group consisting of H, C₁-C₄ alkyl, hydroxy-(C₁-C₂ alkyl), carboxy-(C₁-C₂ alkyl), amino-(C₃-C₅ alkyl), guanidino-(C₂-C₄ alkyl), carbamoyl-(C₁-C₂ alkyl), mercapto-(C₁-C₂ alkyl), methylthio-(C₁-C₃ alkyl), indolylmethyl, phenyl-(C₁-C₂ alkyl), and hydroxyphenyl-(C₁-C₂ alkyl).

95. The compound of claim 83 in which R¹ is CH₂, R² is N, and R³, R²¹, and R²² are members selected from the group consisting of H, C₁-C₄ alkyl, hydroxy-(C₁-C₂ alkyl), carboxy-(C₁-C₂ alkyl), amino-(C₃-C₅ alkyl), guanidino-(C₂-C₄ alkyl), carbamoyl-(C₁-C₂ alkyl), mercapto-(C₁-C₂ alkyl), methylthio-(C₁-C₃ alkyl), indolylmethyl, phenyl-(C₁-C₂ alkyl), and hydroxyphenyl-(C₁-C₂ alkyl).

96. The compound of claim 83 which is a member selected from the group consisting of compounds of the formula 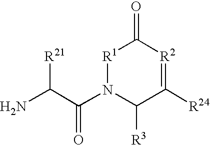
in which R²⁴ is a member selected from the group consisting of an activated leaving group, OR²⁵ where R²⁵ is H or an oxygen-protecting group, SR²⁶ where R²⁶ is H or an alkyl or aryl group, or NR²⁷₂ where the R²⁷'s are members independently selected from the group consisting of H, alkyl, or aryl; and amine-protected analogs of said compounds.

97. The compound of claim 83 which is a member selected from the group consisting of compounds of the formula 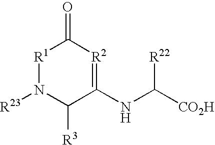
in which R²³ is an amine protecting group, and carboxy-protected analogs of said compounds.

98. The compound of claim 83 which is a member selected from the group consisting of compounds of the formula 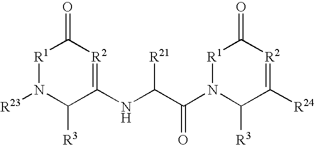
in which R²³ is an amine protecting group and R²⁴ is a member selected from the group consisting of an activated leaving group, OR²⁵ where R²⁵ is H or an oxygen-protecting group, SR²⁶ where R²⁶ is H or an alkyl or aryl group, or NR(²⁷)₂ where each R²⁷ is a member independently selected from the group consisting of H, alkyl, or aryl; and amine-protected analogs of said compounds.

99. The compound of claim 83 which is a member selected from the group consisting of compounds of the formula 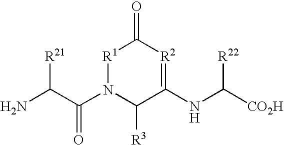
amine-protected analogs of said compounds, carboxy-protected analogs of said compounds, amine-protected and carboxy-protected analogs of said compounds, and amine-protected and carboxy-activated analogs of said compounds.

100. A method for inhibiting the association of a selected peptide with other peptides, said method comprising contacting said selected peptide with a peptide analog defined as a peptide in which at least one amino acid, but less than all amino acids is replaced by an azacyclohexenone group having the formula 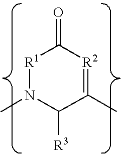
in which:
R¹ is CH₂ or NH,
R² is CH or N, and
R³ is H or an amino acid side chain,
such that in at least one such azacyclohexenone group:
when R¹ is CH₂ and R² is CH, R³ is an amino acid side chain, and
when either R¹ is NH, or R² is N, or R¹ is NH and R² is N, R³ is H or an amino acid side chain,
and when said peptide analog contains two or more azacyclohexenone groups of said formula, R¹, R², and R³ of any one azacyclohexenone group in said peptide analog are either the same as or different from R¹, R², and R³ of any other azacyclohexenone group in said peptide analog,
to achieve a β-sheet like interaction between said selected peptide and said peptide analog.

101. A method for inhibiting the association of a selected peptide with other peptides, said method comprising contacting said selected peptide with a peptide analog defined as a peptide in which at least one amino acid, but less than all amino acids is replaced by an azacyclohexenone group having the formula 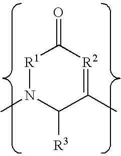
in which:
R¹ is CH₂ or NH,
R² is CH or N, and
when R¹ is CH₂ and R² is CH, R³ is an amino acid side chain, and
when either R is NH, or R² is N, or R¹ is NH and R² is N, R³ is H or an amino acid side chain,
and when said peptide analog contains two or more azacyclohexenone groups of said formula, R¹, R², and R³ of any one azacyclohexenone group in said peptide analog are either the same as or different from R¹, R², and R³ of any other azacyclohexenone group in said peptide analog,
to achieve a β-sheet like interaction between said selected peptide and said peptide analog.

102. The method of claim 101 in which R¹ is CH₂ and R² is N.

103. The method of claim 101 in which R¹ is NH and R² is CH.

104. The method of claim 101 in which R¹ is NH and R² is N.

105. The method of claim 101 in which R¹ is CH₂ and R² is CH.

106. The method of claim 101 in which said azacyclohexenone group is an L-stereoisomer relative to R³ when R³ is an amino acid side chain.

107. The method of claim 101 in which R³ is a member selected from the group consisting of C₁-C₆ alkyl, C₁-C₆ alkyl interrupted by —O—, C₁-C₆ alkyl interrupted by —S—, hydroxy-(C₁-C₆ alkyl), carboxy-(C₁-C₆ alkyl), amino-(C₁-C₆ alkyl), guanidino-(C₁-C₆ alkyl), carbamoyl-(C₁-C₆ alkyl), mercapto-(C₁-C₆ alkyl), indolyl-(C₁-C₃ alkyl), phenyl-(C₁-C₃ alkyl), hydroxyphenyl-(C₁-C₆ alkyl), halophenyl-(C₁-C₆ alkyl), imidazolyl-(C₁-C₆ alkyl), phenyl, and sulfoximino-(C₁-C₆ alkyl).

108. The method of claim 101 in which R³ is a member selected from the group consisting of C₁-C₄ alkyl, hydroxy-(C₁-C₂ alkyl), carboxy-(C₁-C₂ alkyl), amino-(C₃-C₅ alkyl), guanidino-(C₂-C₄ alkyl), carbamoyl-(C₁-C₂ alkyl), mercapto-(C₁-C₂ alkyl), methylthio-(C₁-C₃ alkyl), indolylmethyl, phenyl-(C₁-C₂ alkyl), and hydroxyphenyl-(C₁-C₂ alkyl).

109. The method of claim 101 in which R¹ is CH₂, R² is N, and R³ is a member selected from the group consisting of C₁-C₄ alkyl, hydroxy-(C₁-C₂ alkyl), carboxy-(C₁-C₂ alkyl), amino-(C₃-C₅ alkyl), guanidino-(C₂-C₄ alkyl), carbamoyl-(C₁-C₂ alkyl), mercapto-(C₁-C₂ alkyl), methylthio-(C₁-C₃ alkyl), indolylmethyl, phenyl-(C₁-C₂ alkyl), and hydroxyphenyl-(C₁-C₂ alkyl).

110. The method of claim 101 in which said peptide analog is a peptide in which two or more non-adjacent amino acids are replaced by azacyclohexenone groups of said formula.

111. The method of claim 101 in which said peptide analog is a peptide in which, in at least a portion thereof, every second amino acid is replaced by an azacyclohexenone group of said formula, and the number of said azacyclohexenone groups in said peptide analog is two or more.

112. The method of claim 101 in which the total number of amino acids and azacyclohexenone groups in said peptide analog is from 3 to 200.

113. The method of claim 101 in which the total number of amino acids and azacyclohexenone groups in said peptide analog is from 4 to 20.

114. A method for inhibiting the association of a peptide with a double stranded nucleic acid, said method comprising contacting said peptide with a peptide analog defined as a peptide in which at least one amino acid, but less than all amino acids, is replaced by an azacyclohexenone group having the formula 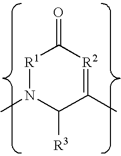
in which:
R¹ is CH₂ or NH,
R² is CH or N, and
R³ is H or an amino acid side chain,
such that in at least one such azacyclohexenone group:
when R¹ is CH₂ and R² is CH, R³ is an amino acid side chain, and
when either R¹ is NH, or R² is N, or R is NH and R² is N, R³ is H or an amino acid side chain,
and when said peptide analog contains two or more azacyclohexenone groups of said formula, R¹, R², and R³ of any one azacyclohexenone group in said peptide analog are either the same as or different from R¹, R², and R³ of any other azacyclohexenone group in said peptide analog,
to achieve a β-sheet-like interaction between said peptide and said peptide analog.

115. A method for inhibiting the association of a peptide with a double stranded nucleic acid, said method comprising contacting said peptide with a peptide analog defined as a peptide in which at least one amino acid, but less than all amino acids, is replaced by an azacyclohexenone group having the formula 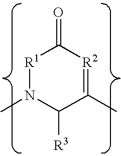
in which:
R¹ is CH₂ or NH,
R² is CH or N, and
when R¹ is CH₂ and R² is CH, R³ is an amino acid side chain, and
when either R¹ is NH, or R² is N, or R is NH and R² is N, R³ is H or an amino acid side chain,
and when said peptide analog contains two or more azacyclohexenone groups of said formula, R¹, R², and R³ of any one azacyclohexenone group in said peptide analog are either the same as or different from R¹, R², and R³ of any other azacyclohexenone group in said peptide analog,
to achieve a β-sheet-like interaction between said peptide and said peptide analog.

116. The method of claim 115 in which R¹ is CH₂ and R² is N.

117. The method of claim 115 in which R¹ is NH and R² is CH.

118. The method of claim 115 in which R¹ is NH and R² is N.

119. The method of claim 115 in which R¹ is CH₂ and R² is CH.

120. The method of claim 115 in which said azacyclohexenone group is an L-stereoisomer relative to R³ when R³ is an amino acid side chain.

121. The method of claim 115 in which R³ is a member selected from the group consisting of C₁-C₆ alkyl, C₁-C₆ alkyl interrupted by —O—, C₁-C₆ alkyl interrupted by —S—, hydroxy-(C₁-C₆ alkyl), carboxy-(C₁-C₆ alkyl), amino-(C₁-C₆ alkyl), guanidino-(C₁-C₆ alkyl), carbamoyl-(C₁-C₆ alkyl), mercapto-(C₁-C₆ alkyl), indolyl-(C₁-C₃ alkyl), phenyl-(C₁-C₃ alkyl), hydroxyphenyl-(C₁-C₆ alkyl), halophenyl-(C₁-C₆ alkyl), imidazolyl-(C₁-C₆ alkyl), phenyl, and sulfoximino-(C₁-C₆ alkyl).

122. The method of claim 115 in which R³ is a member selected from the group consisting of C₁-C₄ alkyl, hydroxy —(C₁-C₂ alkyl), carboxy-(C₁-C₂ alkyl), amino-(C₃-C₅ alkyl), guanidino —(C₂-C₄ alkyl), carbamoyl-(C₁-C₂ alkyl), mercapto-(C₁-C₂ alkyl), methylthio-(C₁-C₃ alkyl), indolylmethyl, phenyl-(C₁-C₂ alkyl), and hydroxyphenyl-(C₁-C₂ alkyl).

123. The method of claim 115 in which R¹ is CH₂, R² is N, and R³ is a member selected from the group consisting of C₁-C₄ alkyl, hydroxy —(C₁-C₂ alkyl), carboxy-(C₁-C₂ alkyl), amino-(C₃-C₅ alkyl), guanidino —(C₂-C₄ alkyl), carbamoyl-(C₁-C₂ alkyl), mercapto-(C₁-C₂ alkyl), methylthio-(C₁-C₃ alkyl), indolylmethyl, phenyl-(C₁-C₂ alkyl), and hydroxyphenyl-(C₁-C₂ alkyl).

124. The method of claim 115 in which said peptide analog is a peptide in which two or more non-adjacent amino acids are replaced by azacyclohexenone groups of said formula.

125. The method of claim 115 in which said peptide analog is a peptide in which, in at least a portion thereof, every second amino acid is replaced by an azacyclohexenone group of said formula, and the number of said azacyclohexenone groups in said peptide analog is two or more.

126. The method of claim 115 in which the total number of amino acids and azacyclohexenone groups in said peptide analog is from 3 to 200.

127. The method of claim 115 in which the total number of amino acids and azacyclohexenone groups in said peptide analog is from 4 to 20.

128. A method for inhibiting the biological activity of a peptide, said method comprising contacting said peptide with a peptide analog defined as a peptide in which at least one amino acid, but less than all amino acids, is replaced by an azacyclohexenone group having the formula 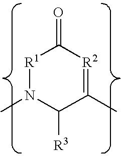
in which:
R¹ is CH₂ or NH,
R² is CH or N, and
R³ is H or an amino acid side chain,
such that in at least one such azacyclohexenone group:
when R¹ is CH₂ and R² is CH, R³ is an amino acid side chain, and
when either R¹ is NH, or R² is N, or R¹ is NH and R² is N, R³ is H or an amino acid side chain,
and when said peptide analog contains two or more azacyclohexenone groups of said formula, R¹, R², and R³ of any one azacyclohexenone group in said peptide analog are either the same as or different from R¹, R², and R³ of any other azacyclohexenone group in said peptide analog,
to achieve a β-sheet-like interaction between said peptide and said peptide analog.

129. A method for inhibiting the biological activity of a peptide, said method comprising contacting said peptide with a peptide analog defined as a peptide in which at least one amino acid, but less than all amino acids, is replaced by an azacyclohexenone group having the formula 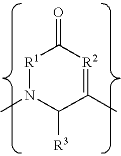
in which:
R¹ is CH₂ or NH,
R² is CH or N, and
when R¹ is CH₂ and R² is CH, R³ is an amino acid side chain, and
when either R¹ is NH, or R² is N, or R¹ is NH and R² is N, R³ is H or an amino acid side chain,
and when said peptide analog contains two or more azacyclohexenone groups of said formula, R¹, R², and R³ of any one azacyclohexenone group in said peptide analog are either the same as or different from R¹, R², and R³ of any other azacyclohexenone group in said peptide analog,
to achieve a β-sheet-like interaction between said peptide and said peptide analog.

130. The method of claim 129 in which R¹ is CH₂ and R² is N.

131. The method of claim 129 in which R¹ is NH and R² is CH.

132. The method of claim 129 in which R¹ is NH and R² is N.

133. The method of claim 129 in which R¹ is CH₂ and R² is CH.

134. The method of claim 129 in which said azacyclohexenone group is an L-stereoisomer relative to R³ when R³ is an amino acid side chain.

135. The method of claim 129 in which R³ is a member selected from the group consisting of C₁-C₆ alkyl, C₁-C₆ alkyl interrupted by —O—, C₁-C₆ alkyl interrupted by —S—, hydroxy —(C₁-C₆ alkyl), carboxy-(C₁-C₆ alkyl), amino-(C₁-C₆ alkyl), guanidino —(C₁-C₆ alkyl), carbamoyl-(C₁-C₆ alkyl), mercapto-(C₁-C₆ alkyl), indolyl-(C₁-C₃ alkyl), phenyl-(C₁-C₃ alkyl), hydroxyphenyl-(C₁-C₆ alkyl), halophenyl-(C₁-C₆ alkyl), imidazolyl-(C₁-C₆ alkyl), phenyl, and sulfoximino-(C₁-C₆ alkyl).

136. The method of claim 129 in which R³ is a member selected from the group consisting of C₁-C₄ alkyl, hydroxy —(C₁-C₂ alkyl), carboxy-(C₁-C₂ alkyl), amino-(C₃-C₅ alkyl), guanidino —(C₂-C₄ alkyl), carbamoyl-(C₁-C₂ alkyl), mercapto-(C₁-C₂ alkyl), methylthio-(C₁-C₃ alkyl), indolylmethyl, phenyl-(C₁-C₂ alkyl), and hydroxyphenyl-(C₁-C₂ alkyl).

137. The method of claim 129 in which R¹ is CH₂, R² is N, and R³ is a member selected from the group consisting of C₁-C₄ alkyl, hydroxy —(C₁-C₂ alkyl), carboxy-(C₁-C₂ alkyl), amino-(C₃-C₅ alkyl), guanidino —(C₂-C₄ alkyl), carbamoyl-(C₁-C₂ alkyl), mercapto-(C₁-C₂ alkyl), methylthio-(C₁-C₃ alkyl), indolylmethyl, phenyl-(C₁-C₂ alkyl), and hydroxyphenyl-(C₁-C₂ alkyl).

138. The method of claim 129 in which said peptide analog is a peptide in which two or more non-adjacent amino acids are replaced by azacyclohexenone groups of said formula.

139. The method of claim 129 in which said peptide analog is a peptide in which, in at least a portion thereof, every second amino acid is replaced by an azacyclohexenone group of said formula, and the number of said azacyclohexenone groups in said peptide analog is two or more.

140. The method of claim 129 in which the total number of amino acids and azacyclohexenone groups in said peptide analog is from 3 to 200.

141. The method of claim 129 in which the total number of amino acids and azacyclohexenone groups in said peptide analog is from 4 to 20.

142. A method for increasing the tendency of a target peptide or a portion of a target peptide to assume a β-strand conformation, said method comprising contacting said target peptide with a peptide analog defined as a peptide in which at least one amino acid, but less than all amino acids, is replaced by an azacyclohexenone group having the formula 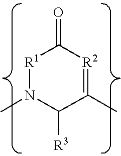
in which:
R¹ is CH₂ or NH,
R² is CH or N, and
R³ is H or an amino acid side chain,
such that in at least one such azacyclohexenone group:
when R¹ is CH₂ and R² is CH, R is an amino acid side chain, and
when either R¹ is NH, or R² is N, or R¹ is NH and R² is N, R³ is H or an amino acid side chain,
and when said peptide analog contains two or more azacyclohexenone groups of said formula, R¹, R², and R³ of any one azacyclohexenone group in said peptide analog are either the same as or different from R¹, R², and R³ of any other azacyclohexenone group in said peptide analog,
to achieve a β-sheet-like interaction between said peptide and said peptide analog.

143. A method for increasing the tendency of a target peptide or a portion of a target peptide to assume a β-strand conformation, said method comprising contacting said target peptide with a peptide analog defined as a peptide in which at least one amino acid, but less than all amino acids, is replaced by an azacyclohexenone group having the formula 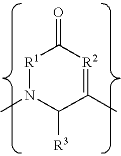
in which:
R¹ is CH₂ or NH,
R² is CH or N, and
when R¹ is CH₂ and R² is CH, R³ is an amino acid side chain, and
when either R¹ is NH, or R² is N, or R¹ is NH and R² is N, R³ is H or an amino acid side chain,
and when said peptide analog contains two or more azacyclohexenone groups of said formula, R¹, R², and R³ of any one azacyclohexenone group in said peptide analog are either the same as or different from R¹, R², and R³ of any other azacyclohexenone group in said peptide analog,
to achieve a β-sheet-like interaction between said peptide and said peptide analog.

144. The method of claim 143 in which R¹ is CH₂ and R² is N.

145. The method of claim 143 in which R¹ is NH and R² is CH.

146. The method of claim 143 in which R¹ is NH and R² is N.

147. The method of claim 143 in which R¹ is CH₂ and R² is CH.

148. The method of claim 143 in which said azacyclohexenone group is an L-stereoisomer relative to R³ when R³ is an amino acid side chain.

149. The method of claim 143 in which R³ is a member selected from the group consisting of C₁-C₆ alkyl, C₁-C₆ alkyl interrupted by —O—, C₁-C₆ alkyl interrupted by —S—, hydroxy-(C₁-C₆ alkyl), carboxy-(C₁-C₆ alkyl), amino-(C₁-C₆ alkyl), guanidino-(C₁-C₆ alkyl), carbamoyl-(C₁-C₆ alkyl), mercapto-(C₁-C₆ alkyl), indolyl-(C₁-C₃ alkyl), phenyl-(C₁-C₃ alkyl), hydroxyphenyl-(C₁-C₆ alkyl), halophenyl-(C₁-C₆ alkyl), imidazolyl-(C₁-C₆ alkyl), phenyl, and sulfoximino-(C₁-C₆ alkyl).

150. The method of claim 143 in which R³ is a member selected from the group consisting of C₁-C₄ alkyl, hydroxy —(C₁-C₂ alkyl), carboxy-(C₁-C₂ alkyl), amino-(C₃-C₅ alkyl), guanidino —(C₂-C₄ alkyl), carbamoyl-(C₁-C₂ alkyl), mercapto-(C₁-C₂ alkyl), methylthio-(C₁-C₃ alkyl), indolylmethyl, phenyl-(C₁-C₂ alkyl), and hydroxyphenyl-(C₁-C₂ alkyl).

151. The method of claim 143 in which R¹ is CH₂, R² is N, and R³ is a member selected from the group consisting of C₁-C₄ alkyl, hydroxy —(C₁-C₂ alkyl), carboxy-(C₁-C₂ alkyl), amino-(C₃-C₅ alkyl), guanidino —(C₂-C₄ alkyl), carbamoyl-(C₁-C₂ alkyl), mercapto-(C₁-C₂ alkyl), methylthio-(C₁-C₃ alkyl), indolylmethyl, phenyl-(C₁-C₂ alkyl), and hydroxyphenyl-(C₁-C₂ alkyl).

152. The method of claim 143 in which said peptide analog is a peptide in which two or more non-adjacent amino acids are replaced by azacyclohexenone groups of said formula.

153. The method of claim 143 in which said peptide analog is a peptide in which, in at least a portion thereof, every second amino acid is replaced by an azacyclohexenone group of said formula, and the number of said azacyclohexenone groups in said peptide analog is two or more.

154. The method of claim 143 in which the total number of amino acids and azacyclohexenone groups in said peptide analog is from 3 to 200.

155. The method of claim 143 in which the total number of amino acids and azacyclohexenone groups in said peptide analog is from 4 to 20.

156. A method for extracting a target peptide having a selected amino acid sequence from a mixture of peptides, said method comprising contacting said mixture with a capture peptide that is covalently bonded to a solid support and associates with said amino acid sequence in a β-sheet interaction, said capture peptide comprising amino acids and at least one azacyclohexenone group having the formula 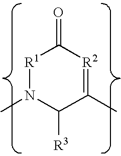
in which:
R¹ is CH₂ or NH,
R² is CH or N, and
R³ is H or an amino acid side chain,
such that in at least one such azacyclohexenone group:
when R¹ is CH₂ and R² is CH, R³ is an amino acid side chain, and
when either R¹ is NH, or R² is N, or R¹ is NH and R² is N, R³ is H or an amino acid side chain,
and when said peptide analog contains two or more azacyclohexenone groups of said formula, R¹, R², and R³ of any one azacyclohexenone group in said peptide analog are either the same as or different from R¹, R², and R³ of any other azacyclohexenone group in said peptide analog,
to achieve a β-sheet-like interaction between said target peptide and said capture analog.

157. A method for extracting a target peptide having a selected amino acid sequence from a mixture of peptides, said method comprising contacting said mixture with a capture peptide that is covalently bonded to a solid support and associates with said amino acid sequence in a β-sheet interaction, said capture peptide comprising amino acids and at least one azacyclohexenone group having the formula 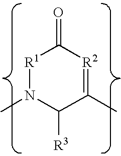
in which:
R¹ is CH₂ or NH,
R² is CH or N, and
when R¹ is CH₂ and R² is CH, R³ is an amino acid side chain, and
when either R¹ is NH, or R² is N, or R¹ is NH and R² is N, R³ is H or an amino acid side chain,
and when said peptide analog contains two or more azacyclohexenone groups of said formula, R¹, R², and R³ of any one azacyclohexenone group in said peptide analog are either the same as or different from R¹, R², and R³ of any other azacyclohexenone group in said peptide analog,
to achieve a β-sheet-like interaction between said target peptide and said capture analog.

158. The method of claim 157 in which R¹ is CH₂ and R² is N.

159. The method of claim 157 in which R¹ is NH and R² is CH.

160. The method of claim 157 in which R¹ is NH and R² is N.

161. The method of claim 157 in which R¹ is CH₂ and R² is CH.

162. The method of claim 157 in which said azacyclohexenone group is an L-stereoisomer relative to R³ when R³ is an amino acid side chain.

163. The method of claim 157 in which R³ is a member selected from the group consisting of C₁-C₆ alkyl, C₁-C₆ alkyl interrupted by —O—, C₁-C₆ alkyl interrupted by —S—, hydroxy-(C₁-C₆ alkyl), carboxy-(C₁-C₆ alkyl), amino-(C₁-C₆ alkyl), guanidino-(C₁-C₆ alkyl), carbamoyl-(C₁-C₆ alkyl), mercapto-(C₁-C₆ alkyl), indolyl-(C₁-C₃ alkyl), phenyl-(C₁-C₃ alkyl), hydroxyphenyl-(C₁-C₆ alkyl), halophenyl-(C₁-C₆ alkyl), imidazolyl-(C₁-C₆ alkyl), phenyl, and sulfoximino-(C₁-C₆ alkyl).

164. The method of claim 157 in which R³ is a member selected from the group consisting of C₁-C₄ alkyl, hydroxy-(C₁-C₂ alkyl), carboxy-(C₁-C₂ alkyl), amino-(C₃-C₅ alkyl), guanidino-(C₂-C₄ alkyl), carbamoyl-(C₁-C₂ alkyl), mercapto-(C₁-C₂ alkyl), methylthio-(C₁-C₃ alkyl), indolylmethyl, phenyl-(C₁-C₂ alkyl), and hydroxyphenyl-(C₁-C₂ alkyl).

165. The method of claim 157 in which R¹ is CH₂, R² is N, and R³ is a member selected from the group consisting of C₁-C₄ alkyl, hydroxy-(C₁-C₂ alkyl), carboxy-(C₁-C₂ alkyl), amino-(C₃-C₅ alkyl), guanidino-(C₂-C₄ alkyl), carbamoyl-(C₁-C₂ alkyl), mercapto-(C₁-C₂ alkyl), methylthio-(C₁-C₃ alkyl), indolylmethyl, phenyl-(C₁-C₂ alkyl), and hydroxyphenyl-(C₁-C₂ alkyl).

166. The method of claim 157 in which said capture peptide is a peptide in which two or more non-adjacent amino acids are replaced by azacyclohexenone groups of said formula.

167. The method of claim 157 in which said capture peptide is a peptide in which, in at least a portion thereof, every second amino acid is replaced by an azacyclohexenone group of said formula, and the number of said azacyclohexenone groups in said peptide analog is two or more.

168. The method of claim 157 in which the total number of amino acids and azacyclohexenone groups in said capture peptide is from 3 to 200.

169. The method of claim 157 in which the total number of amino acids and azacyclohexenone groups in said capture peptide is from 4 to 20.
